SEQUENCE LISTING (2 sequences)

SEQ ID NO: 1 - DNA, 40 bp, synthetic construct
  source 1..40: mol_type = other DNA
  modified_base 1: mod_base = OTHER; note = 5' acrydite modified nucleotide
  modified_base 21: mod_base = OTHER; note = Locked nucleic acid
  modified_base 23: mod_base = OTHER; note = Locked nucleic acid
  modified_base 25: mod_base = OTHER; note = Locked nucleic acid
  modified_base 27: mod_base = OTHER; note = Locked nucleic acid
  modified_base 29: mod_base = OTHER; note = Locked nucleic acid
  modified_base 31: mod_base = OTHER; note = Locked nucleic acid
  modified_base 33: mod_base = OTHER; note = Locked nucleic acid
  modified_base 35: mod_base = OTHER; note = Locked nucleic acid
  modified_base 37: mod_base = OTHER; note = Locked nucleic acid
  modified_base 39: mod_base = OTHER; note = Locked nucleic acid
ttgagtggatggagtgtaattttttttttttttttttttt

SEQ ID NO: 2 - DNA, 20 bp, synthetic construct
  source 1..20: mol_type = other DNA
  misc_difference 1..20: note = This sequence may encompass 5-20 nucleotides
tttttttttttttttttttt

(19) United States
(12) Patent Application Publication
(10) Pub. No.: US 2026/0301438 A1
(43) Pub. Date: Oct. 1, 2026

(54) THREE-DIMENSIONAL IMAGING SYSTEMS AND METHODS FOR DETERMINING NUCLEIC ACIDS IN THICK TISSUES

(71) Applicant: President and Fellows of Harvard College, Cambridge, MA (US)

(72) Inventors: Xiaowei Zhuang, Cambridge, MA (US); Rongxin Fang, Cambridge, MA (US); Aaron Halpern, Cambridge, MA (US)

(73) Assignee: President and Fellows of Harvard College, Cambridge, MA (US)

(21) Appl. No.: 19/489,937

(22) PCT Filed: Jun. 4, 2024
(86) PCT No.: PCT/US2024/032412

§ 371 Date: Dec. 4, 2025

Related U.S. Application Data
(60) Provisional application No. 63/506,283, filed on Jun. 5, 2023.

(51) Int. Cl. G06V 20/69; C12Q 1/6841; G02B 21/00; G06V 10/82; G06V 20/64

(52) U.S. Cl. CPC G06V 20/693; C12Q 1/6841; G02B 21/0044; G02B 21/008; G06V 10/82; G06V 20/64; G06V 20/695; G06V 20/698; G06V 2201/03

(57) ABSTRACT

The present disclosure generally relates to microscopy, including confocal microscopy. In some cases, techniques such as MERFISH can be used to determine nucleic acids within a sample, with images acquired using confocal microscopy. In some cases, the nucleic acids may be determined in 3 dimensions. In some cases, relatively thick samples, e.g., at least 100 micrometers thick, may be determined. In some embodiments, deep learning or other machine learning techniques may be used to enhance the image quality and/or speed up the confocal imaging process.

### RELATED APPLICATIONS

This application claims the benefit of U.S. Provisional Patent Application Ser. No. 63/506,283, filed Jun. 5, 2023, entitled “Three-Dimensional Single-Cell Transcriptome Imaging of Thick Tissues,” by Zhuang, et al., incorporated herein by reference in its entirety.

### REFERENCE TO AN ELECTRONIC SEQUENCE LISTING

The contents of the electronic sequence listing (H049870798WO00-SEQ-TC.xml; Size: 9,274 bytes; and Date of Creation: Jun. 4, 2024) is herein incorporated by reference in its entirety.

### FIELD

The present disclosure generally relates to microscopy including confocal microscopy and spatial genomics.

### BACKGROUND

Multiplexed error-robust fluorescence in-situ hybridization (MERFISH) allows genome-scale imaging of nucleic acids such as RNA, DNA as well as epigenetic elements in individual cells in intact tissues. To date, MERFISH has been applied to image relatively thin tissue samples.

Recent advances in genome-scale imaging methods have allowed in situ gene expression profiling, 3D-genome imaging, and epigenomic profiling of individual cells, which has in turn allowed identification and spatial mapping of molecularly defined cell types in intact tissues. Among these methods, multiplexed error-robust fluorescence in situ hybridization (MERFISH) allows simultaneously imaging of thousands of genes by using combinatorial labeling to assign unique barcodes to individual genes, sequential rounds of imaging to read out the barcodes of individual RNA molecules, and error-robust barcoding schemes to ensure high detection accuracy. MERFISH has also been extended to allow spatially resolved 3D-gemone imaging and epigenomic profiling of individual cells. See, e.g., U.S. Pat. No. 11,098,303, incorporated herein by reference.

MERFISH measurements have been performed on thin tissue sections of ~10 micrometer thickness. However, tissue deformation that occurs during sectioning makes it challenging to align images of serial thin sections and determine the 3D molecular and cellular architecture of tissues. In addition, it is difficult to align thin-section MERFISH images with images of other cellular properties, such as cell morphology and neuronal activity acquired through thick-tissue or live-animal imaging, which makes multi-modal characterizations of molecular, morphological, and functional properties of cells challenging. Thus, it is highly desirable to extend MERFISH to thick-tissue imaging. However, thick-tissue MERFISH imaging faces several challenges. First, the fluorescence background caused by out-of-focus signal reduces image quality. Second, spherical aberration introduced by refractive index mismatch leads to degraded image resolution and quality in the deeper part of the thick samples. Third, the MERFISH protocol previously optimized for thin-specimen imaging is not adequate for imaging thick samples. Accordingly, improvements are still needed.

### SUMMARY

The present disclosure generally relates to microscopy including confocal microscopy. The subject matter of the present disclosure involves, in some cases, interrelated products, alternative solutions to a particular problem, and/or a plurality of different uses of one or more systems and/or articles.

One aspect is generally directed to using a confocal microscope to acquire images of a sample, and determining nucleic acids within the sample using MERFISH.

Another aspect is generally directed to using a confocal microscope to acquire images of a sample, and determining nucleic acids within the sample in 3 dimensions using the images.

Yet another aspect is generally directed to exposing a sample to a plurality of nucleic acid probes; for each of the nucleic acid probes, determining binding of the nucleic acid probes within the sample by acquiring images of the sample using a confocal microscope; creating codewords based on the binding of the nucleic acid probes; and for at least some of the codewords, matching the codeword to a valid codeword wherein, if no match is found, applying error correction to the codeword to form a valid codeword.

Yet another aspect is generally directed to using a confocal microscope to acquire images of a sample, enhancing the images using machine learning, and determining nucleic acids within the sample.

Still another aspect is generally directed to using a confocal microscope to acquire images of a sample, enhancing the images using machine learning, and determining nucleic acids within the sample using MERFISH.

Another aspect is generally directed to using a confocal microscope to acquire images of a sample, and determining nucleic acids within the sample by exposing the sample to a plurality of nucleic acid probes and determining binding of the plurality of the nucleic acid probes to the sample.

Yet another aspect is generally directed to using a confocal microscope to acquire images of a sample, enhancing the images using machine learning, and determining nucleic acids within the sample by exposing the sample to a plurality of nucleic acid probes and determining binding of the plurality of the nucleic acid probes to the sample.

In another aspect, the present disclosure encompasses methods of making one or more of the embodiments described herein. In still another aspect, the present disclosure encompasses methods of using one or more of the embodiments described herein.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1D illustrate enhanced performance of confocal MERFISH imaging by deep learning, in accordance with one embodiment;

FIGS. 2A-2G illustrate imaging of thick brain tissue sections, in another embodiment;

FIGS. 3A-3F illustrate spatial organization of cell types in tissue sections, in yet another embodiment;

FIG. 4 illustrates a comparison of epifluorescece and confocal images, in still another embodiment;

FIGS. 5A-5D illustrate MERFISH images of RNA molecules in tissues, in yet another embodiment;

FIGS. 6A-6E illustrate MERFISH encoding and readouts, in still another embodiment;

FIGS. 7A-7D illustrate displacement of RNA molecules, in yet another embodiment;

FIGS. 8A-8C illustrates gel expansion, in one embodiment;

FIGS. 9A-9C illustrates 3D MERFISH imaging in mouse tissue, in another embodiment;

FIG. 10 illustrates cell type identification in mouse tissue, in one embodiment;

FIGS. 11A-11B illustrate MERFISH imaging in mouse tissue, in another embodiment;

FIGS. 12A-12B illustrate cell type identification in mouse tissue, in still another embodiment; and

FIGS. 13A-13B illustrates multiple read sequences distributed in a population of different nucleic acid probes, in accordance with another embodiment.

### DETAILED DESCRIPTION

The present disclosure generally relates to microscopy, including confocal microscopy. In some cases, techniques such as MERFISH can be used to determine nucleic acids within a sample, with images acquired using confocal microscopy. In some cases, the nucleic acids may be determined in 3 dimensions. In some cases, relatively thick samples, e.g., greater than 10 micrometers thick or greater than 100 micrometers thick, may be determined. In some embodiments, deep learning or other machine learning techniques may be used to enhance the image quality and/or speed up the confocal imaging process.

One aspect is generally drawn to systems and methods for imaging or determining nucleic acids within a cell or other sample. The nucleic acids may be determined in 2 or 3 dimensions. In some embodiments, the transcriptome of a cell may be determined. Certain embodiments are directed to determining nucleic acids, such as mRNA, within cells at relatively high resolutions. In some embodiments, a plurality of nucleic acid probes may be applied to a sample, and their binding within the sample determined, e.g., using confocal microscopy, to determine locations of the nucleic acid probes within the sample.

The sample may include a cell culture, a suspension of cells, a biological tissue, a biopsy, an organism, or the like. The sample may also be cell-free but nevertheless contain nucleic acids. If the sample contains a cell, the cell may be a human cell, or any other suitable cell, e.g., a mammalian cell, a fish cell, an insect cell, a plant cell, or the like. More than one cell may be present in some cases.

Confocal microscopy can be used, in certain aspects, to analyze relatively thick samples, e.g., samples having a thickness of at least 10 micrometers, at least 20 micrometers, at least 30 micrometers, at least 40 micrometers, at least 50 micrometers, at least 60 micrometers, at least 70 micrometers, at least 80 micrometers, at least 90 micrometers, at least 100 micrometers, at least 110 micrometers, at least 125 micrometers, at least 150 micrometers, at least 175 micrometers, at least 200 micrometers, at least 225 micrometers, at least 250 micrometers, at least 300 micrometers, at least 350 micrometers, at least 400 micrometers, at least 450 micrometers, at least 500 micrometers, etc.

In some instances, confocal microscopy uses a spatial pinhole, a spinning disk, or other techniques to block out-of-focus light in image formation. In some embodiments, capturing images at different focal depths in a sample may allow for the reconstruction of three-dimensional images of the sample. For instance, different images can be obtained of a sample with different depths. In some cases, the images may be spaced apart (e.g., in the z-axis) by at least 1 micrometer, at least 2 micrometers, at least 3 micrometers, at least 4 micrometers, at least 5 micrometers, at least 6 micrometers, at least 7 micrometers, at least 8 micrometers, at least 9 micrometers, at least 10 micrometers, at least 12 micrometers, at least 15 micrometers, at least 20 micrometers, etc.

A variety of confocal microscopes can be used according to various embodiments. For instance, the confocal microscopy may be a laser scanning confocal microscope, a spinning disk confocal microscope, a dual spinning-disk confocal microscope, a programmable array microscope (e.g., that may use an electronically controlled spatial light modulator to produce a set of moving pinholes), or the like.

In addition, in some aspects, one or more images that are acquired can be enhanced using machine learning or artificial intelligence techniques such as deep learning, graph convolutional networks, reinforcement learning, neural networks, recurrent neural networks, or the like. In some cases, machine learning techniques may involve training a machine learning model on a training set of images, e.g., acquired as discussed herein, then using the trained machine learning model to enhance images. In some cases, a model may be trained using a data set having suitable properties (for example, precision, clarity of image, edges in image, color, shape, etc.). In some cases, the training data may be labelled, for example, to allow for supervised learning, unsupervised learning, reinforcement techniques, or the like. For example, one or images may be enhanced by reducing blur, increase edging definition, improving boundary definition, improving contrast, or the like. In some embodiments, the output image may have enhanced properties or image segments with enhanced properties. Examples include, but are not limited to, the enhancement of signal-to-noise ratio or signal-to-background ratio. In some cases, the trained machine learning model can be updated, e.g., based on evaluation of the enhanced images. In some embodiments, the output of the machine learning model (e.g., one or more enhanced images) may be evaluated against the target object (e.g., nucleic acid localization). In some embodiments, updating the trained machine learning model may be performed via reinforcement learning, or other techniques. In addition, in some cases, a pre-trained model may be used that is able to take an input and produce an enhanced image output.

Various methods or processes outlined herein may be coded as software that is executable on one or more processors that employ any one of a variety of operating systems or platforms, which may in some cases be used to improve the underlying computer system on which it is implemented. Additionally, such software may be written using any of numerous suitable programming languages and/or programming or scripting tools, and also may be compiled as executable machine language code or intermediate code that is executed on a virtual machine or a suitable framework.

In this respect, various inventive concepts described herein may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments. The non-transitory computer-readable medium or media may be transportable, such that the program or programs stored thereon may be loaded onto any computer resource to implement various embodiments as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods such as discussed herein need not reside on a single computer or processor, but may be distributed in a modular fashion among different computers or processors to implement various embodiments.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

As mentioned, the sample may be immobilized or embedded within a polymer or a gel, partially or completely. In some cases, the sample may be embedded within a relatively large polymer or gel, which can then be sectioned or sliced in some cases to produce smaller portions for analysis, e.g., using various microtomy techniques commonly available to those of ordinary skill in the art. For instance, tissues or organs may be immobilized within a suitable polymer or gel.

A variety of polymers may be used in some embodiments. In some cases, the polymer may be selected to be relatively optically transparent. The polymer may also be one that does not significantly distort during the polymerization process, although in some cases, the polymer may exhibit some distortion. In some cases, the amount of distortion may be determined as a relative change in size that is less than 5, less than 4, less than 3, less than 2, less than 1.5, less than 1.3, or less than 1.2 (i.e., a change in size of 2 means that a sample doubles in linear dimension), or inverses of these (i.e., an inverse change in size of 2 means that a sample halves in linear dimensions).

In some embodiments, the gel may be selected to exhibit relatively low expansion, e.g., under various conditions. For example, the gel may exhibit linear expansion of no more than 10%, no more than 9%, no more than 8%, no more than 7%, no more than 6%, no more than 5%, no more than 4%, no more than 3%, no more than 2%, or no more than 1%, e.g., during use as various buffers are applied to it.

For instance, in some cases, the gel may be prepared at relatively low temperatures, e.g., less than 25° C., less than 20° C., less than 15° C., less than 10° C., less than 8° C., less than 6° C., less than 4° C., less than 2° C., less than 0° C., etc. In addition, in some cases, the gel may be prepared using relatively low concentrations of initiator, e.g., less than 1% vol/vol, less than 0.5% vol/vol, less than 0.2% vol/vol, or less than 0.1% vol/vol.

Non-limiting examples of suitable polymers include polyacrylamide and agarose. In some cases, the polymer is a gel or a hydrogel. A variety of polymers could be used in various embodiments that involve chemical cross links between gel subunits, including but not limited to acrylic acid, acrylamide, ethylene glycol diacrylate, ethylene glycol dimetharcrylate, poly(ethylene glycol dimethacrylate); and/or hydrophobic or hydrogen bonding interactions, such as poly(N-isopropyl acrylamide), methyl cellulose, (ethylene oxide)-(propylene oxide)-(ethylene oxide terpolymers, sodium alginate, poly(vinyl alcohol), alignate, chitosan, gum Arabic, gelatin, and agarose.

In one set of embodiments, anchor probes may be used during the polymerization process. The anchor probes may include a portion that is able to polymerize with the polymer during the polymerization process, and is able to immobilize a target, e.g., chemically and/or physically. For example, in the case of polyacrylamide, the anchor probe may include an acrydite portion that can polymerize and become incorporated into the polymer.

The anchor probe may also contain a portion that can interact with and bind to nucleic acid molecules, or other molecules in which immobilization is desired, e.g., proteins or lipids, other desired targets, etc. The immobilization may be covalent or non-covalent. For example, to immobilize a target nucleic acid, the anchor probe may comprise a nucleic acid comprising an acrydite portion (e.g., at the 5′ end, the 3′ end, an internal base, etc.) and a nucleic acid sequence substantially complementary to at least a portion of the target nucleic acid. For instance, the nucleic acid may be complementary to at least 5, 6, 7, 8, 9, 10, 11, 12, 13, 14, 15, 16, 17, 18, 19, or 20 or more nucleotides of the nucleic acid. In some cases the complementarity may be exact (Watson-Crick complementarity), or there may be 1, 2, or more mismatches. In some cases, the anchor probe can be configured to immobilize mRNA, e.g., in the case of transcriptome analysis. For instance, in one set of embodiments, the anchor probe may contain a plurality of thymine nucleotides, e.g., sequentially, for binding to the poly-A tail of an mRNA. Thus, for example, the anchor probe can have at least 5, 6, 7, 8, 9, 10, 11, 12, 13, 14, 15, 16, 17, 18, 19, or 20 or more consecutive thymine nucleotides (SEQ ID NO: 2) (e.g., a poly-dT portion) within the anchor probe. In some cases, at least some of the thymine nucleotides may be “locked” thymine nucleotides. These may comprise at least 20%, at least 30%, at least 40%, at least 50%, at least 60%, at least 70%, or at least 80% of these thymine nucleotides. In certain embodiments, the locked and non-locked nucleotides may alternate. Such locked thymine nucleotides may be useful, for example, to stabilize the hybridization of the poly-A tails of the mRNA with the anchor probe.

Other methods may be used to anchor nucleic acids, or other molecules in which immobilization is desired. In one set of embodiments, nucleic acids such as DNA or RNA may be immobilized by covalent bonding. For example, in one set of embodiments, an alkylating agent may be used that covalently binds to RNA or DNA and contains a second chemical moiety that can be incorporated into the polyacrylamide as it is polymerized. In yet another set of embodiments, the terminal ribose in an RNA molecule may be oxidized using sodium periodate (or another oxidizing agent) to produce an aldehyde, which may be cross-linked to acrylamide, or other polymer or gel. In other embodiments, chemical agents that are able to modify bases may be used, such as aldehydes, e.g. paraformaldehyde or gluteraldehyde, alkylating agents, or succinimidyl-containing groups; chemical agents that modify the terminal phosphate, such as carboiimides, e.g., EDC (1-ethyl-3-(3-dimethylaminopropyl) carbodiimide); chemical agents that modify internal sugars, such as p-maleimido-phenyl isocyanate; or chemical agents that modify terminal sugars, such as sodium periodate. In some cases, these chemical agents can carry a second chemical moiety that can then be directly cross-linked to the gel or polymer, and/or which can be further modified with a compound that can be directly cross linked to the gel or polymer.

In yet other embodiments, a nucleic acid may be immobilized using anchor probes having substantially complementary portions to the DNA or RNA. There may be 5, 6, 7, 8, 9, 10, 12, 13, 14, 15, 20, 25, 30, 35, 40, 45, 50 or more complementary nucleotides between the anchor probe and the nucleic acid. In still another set of embodiments, the nucleic acids may be physically tangled within the polymer or gel, e.g., due to their length, and, thus, unable to diffuse from their original location within the gel.

Similar anchor probes may be used to immobilize other components to a polymer or gel, in other embodiments. For example, in one set of embodiments, an antibody able to specifically bind to a suitable target (e.g., another protein, a lipid, a carbohydrate, a virus, etc.) may be modified to include an acrydite moiety that can become incorporated within a polymer or gel.

In addition, it should be understood that the embedding of the sample within the matrix and the immobilization of nucleic acids (or other desired targets) may be performed in any suitable order in various embodiments. For instance, immobilization may occur before, during, or after embedding of the sample. In some cases, the target may be chemically modified or reacted to cross-link to the gel or polymer before or during formation of the gel or polymer.

After immobilization of nucleic acids, or other suitable molecules, to the polymer or gel, other components within the sample may be “cleared.” Such clearance may include removal of the components, and/or degradation of the components (e.g., to smaller components, components that are not fluorescent, etc.) that are not the desired target. In some cases, at least 50%, at least 60%, at least 70%, at least 80%, or at least 90% of the undesired components within the sample may be cleared. Multiple clearance steps can also be performed in certain embodiments, e.g., to remove various undesired components. As discussed, it is believed that the removal of such components may decrease background during analysis (for example, by decreasing background and/or off-target binding), while desired components (such as nucleic acids) can be immobilized and thus not cleared.

For example, proteins may be cleared from the sample using enzymes, denaturants, chelating agents, chemical agents, and the like, which may break down the proteins into smaller components and/or amino acids. These smaller components may be easier to remove physically, and/or may be sufficiently small or inert such that they do not significantly affect the background. Similarly, lipids may be cleared from the sample using surfactants or the like. In some cases, one or more of these are used, e.g., simultaneously or sequentially. Non-limiting examples of suitable enzymes include proteinases such as proteinase K, proteases or peptidases, or digestive enzymes such as trypsin, pepsin, or chymotrypsin. Non-limiting examples of suitable denaturants include guanidine HCl, acetone, acetic acid, urea, or lithium perchlorate. Non-limiting examples of chemical agents able to denature proteins include solvents such as phenol, chloroform, guanidinium isocyananate, urea, formamide, etc. Non-limiting examples of surfactants include Triton X-100 (polyethylene glycol p-(1,1,3,3-tetramethylbutyl)-phenyl ether), SDS (sodium dodecyl sulfate), Igepal CA-630, or poloxamers. Non-limiting examples of chelating agents include ethylenediaminetetraacetic acid (EDTA), citrate, or polyaspartic acid. In some embodiments, compounds such as these may be applied to the sample to clear proteins, lipids, and/or other components. For instance, a buffer solution (e.g., containing Tris or tris(hydroxymethyl)aminomethane) may be applied to the sample, then removed.

Non-limiting examples of DNA enzymes that may be used to remove DNA include DNase I, dsDNase, a variety of restriction enzymes, etc. Non-limiting examples of techniques to clear RNA include RNA enzymes such as RNase A, RNase T, or RNase H, or chemical agents, e.g., via alkaline hydrolysis (for example, by increasing the pH to greater than 10). Non-limiting examples of systems to remove sugars or extracellular matrix include enzymes such as chitinase, heparinases, or other glycosylases. Non-limiting examples of systems to remove lipids include enzymes such as lipidases, chemical agents such as alcohols (e.g., methanol or ethanol), or detergents such as Triton X-100 or sodium dodecyl sulfate. Many of these are readily available commercially. In this way, the background of the sample may be removed, which may facilitate analysis of the nucleic acid probes or other desired targets, e.g., using fluorescence microscopy, or other techniques as discussed herein. As mentioned, in various embodiments, various targets (e.g., nucleic acids, certain proteins, lipids, viruses, or the like) may be immobilized, while other non-targets may be cleared using suitable agents or enzymes. As a non-limiting example, if a protein (such as an antibody) is immobilized, then RNA enzymes, DNA enzymes, systems to remove lipids, sugars, etc. may be used.

Techniques such as these may be used, in some aspects, to determine nucleic acids in a sample. The nucleic acids to be determined may be, for example, DNA, RNA, epigenetic elements, or other nucleic acids that are present within a cell (or other sample). The nucleic acids may be endogenous to the cell, or added to the cell. For instance, the nucleic acid may be viral, or artificially created. In some cases, the nucleic acid to be determined may be expressed by the cell. The nucleic acid is RNA in some embodiments. The RNA may be coding and/or non-coding RNA. Non-limiting examples of RNA that may be studied within the cell include mRNA, siRNA, rRNA, miRNA, tRNA, lncRNA, snoRNAs, snRNAs, exRNAs, piRNAs, or the like.

In some cases, a significant portion of the nucleic acid within the cell may be studied. For instance, in some cases, enough of the RNA present within a cell may be determined so as to produce a partial or complete transcriptome of the cell. In some cases, at least 4 types of mRNAs are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of mRNAs may be determined within a cell.

In some cases, the transcriptome of a cell may be determined. It should be understood that the transcriptome generally encompasses all RNA molecules produced within a cell, not just mRNA. Thus, for instance, the transcriptome may also include rRNA, tRNA, siRNA, etc. In some embodiments, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

The determination of one or more nucleic acids within the cell or other sample may be qualitative and/or quantitative. In addition, the determination may also be spatial, e.g., the position of the nucleic acid within the cell or other sample may be determined in two or three dimensions. In some embodiments, the positions, number, and/or concentrations of nucleic acids within the cell (or other sample) may be determined.

In some cases, a significant portion of the genome of a cell may be determined. The determined genomic segments may be continuous or interspersed on the genome. For example, in some cases, at least 4 genomic segments are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 genomic segments may be determined within a cell.

In some cases, the entire genome of a cell may be determined. It should be understood that the genome generally encompasses all DNA molecules produced within a cell, not just chromosome DNA. Thus, for instance, the genome may also include, in some cases, mitochondria DNA, chloroplast DNA, plasmid DNA, etc. In some embodiments, at least about 5%, at least about 10%, at least about 15%, at least about 20%, at least about 25%, at least about 30%, at least about 40%, at least about 50%, at least about 60%, at least about 70%, at least about 80%, at least about 90%, or 100% of the genome of a cell may be determined.

In some cases, the epigenome of a cell may be determined. The epigenome may encompass chromatin with chemically modified DNA or chemically modified histones, or chromatin with other DNA-binding proteins. In some embodiments, at least about 5%, at least about 10%, at least about 15%, at least about 20%, at least about 25%, at least about 30%, at least about 40%, at least about 50%, at least about 60%, at least about 70%, at least about 80%, at least about 90%, or 100% of the epigenome of a cell may be determined.

In addition, certain aspects are directed to systems and methods that allow the copy numbers and spatial localizations of thousands of RNA species, genomic segments, and/or epigenetic elements to be determined in single cells. Some of these techniques are called Multiplexed Error-Robust Fluorescence in Situ Hybridization or “MERFISH,” e.g., as would be known to those of ordinary skill in the art. See, e.g., U.S. Pat. No. 11,098,303, incorporated herein by reference in its entirety. In some cases, error correction can also be used. For example, in some embodiments, codewords may be based on the binding of the plurality of nucleic acid probes, and in some cases, the codewords may define an error-correcting code to reduce or prevent misidentification of the nucleic acids. In certain cases, a relatively large number of different targets may be identified using a relatively small number of labels, e.g., by using various combinatorial approaches. Using error-robust encoding schemes may allow the imaging of hundreds to thousands of RNA species, genomic segments, and/or epigenetic elements in a sample.

For example, as discussed herein, a variety of nucleic acid probes may be used to determine one or more nucleic acids within a cell or other sample. The probes may comprise nucleic acids (or entities that can hybridize to a nucleic acid, e.g., specifically) such as DNA, RNA, LNA (locked nucleic acids), PNA (peptide nucleic acids), or combinations thereof. In some cases, additional components may also be present within the nucleic acid probes, e.g., as discussed below. Any suitable method may be used to introduce nucleic acid probes into a cell.

For example, in some embodiments, the cell is fixed prior to introducing the nucleic acid probes, e.g., to preserve the positions of the nucleic acids within the cell. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE).

The nucleic acid probes may be introduced into the cell (or other sample) using any suitable method. In some cases, the cell may be sufficiently permeabilized such that the nucleic acid probes may be introduced into the cell by flowing a fluid containing the nucleic acid probes around the cells. In some cases, the cells may be sufficiently permeabilized as part of a fixation process; in other embodiments, cells may be permeabilized by exposure to certain chemicals such as ethanol, methanol, Triton, or the like. In addition, in some embodiments, techniques such as electroporation or microinjection may be used to introduce nucleic acid probes into a cell or other sample.

Certain embodiments are generally directed to nucleic acid probes that are introduced into a cell (or other sample). The probes may comprise any of a variety of entities that can hybridize to a nucleic acid, typically by Watson-Crick base pairing, such as DNA, RNA, LNA, PNA, etc., depending on the application. The nucleic acid probe typically contains a target sequence that is able to bind to at least a portion of a target nucleic acid, in some cases specifically. When introduced into a cell or other system, the target system may be able to bind to a specific target nucleic acid (e.g., an mRNA, or other nucleic acids as discussed herein). In some cases, the nucleic acid probes may be determined using signaling entities (e.g., as discussed below), and/or by using secondary nucleic acid probes able to bind to the nucleic acid probes (i.e., to primary nucleic acid probes). The determination of such nucleic acid probes is discussed in detail below.

In some cases, more than one type of (primary) nucleic acid probe may be applied to a sample, e.g., simultaneously. For example, there may be at least 2, at least 5, at least 10, at least 25, at least 50, at least 75, at least 100, at least 300, at least 1,000, at least 3,000, at least 10,000, or at least 30,000 distinguishable nucleic acid probes that are applied to a sample, e.g., simultaneously or sequentially.

The target sequence may be positioned anywhere within the nucleic acid probe (or primary nucleic acid probe or encoding nucleic acid probe). The target sequence may contain a region that is substantially complementary to a portion of a target nucleic acid. In some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc. Typically, complementarity is determined on the basis of Watson-Crick nucleotide base pairing.

The target sequence of a (primary) nucleic acid probe may be determined with reference to a target nucleic acid suspected of being present within a cell or other sample. For example, a target nucleic acid to a protein may be determined using the protein's sequence, by determining the nucleic acids that are expressed to form the protein. In some cases, only a portion of the nucleic acids encoding the protein are used, e.g., having the lengths as discussed above. In addition, in some cases, more than one target sequence that can be used to identify a particular target may be used. For instance, multiple probes can be used, sequentially and/or simultaneously, that can bind to or hybridize to different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

In some embodiments, a nucleic acid probe, such as a primary nucleic acid probe, may also comprise one or more “read” sequences. However, it should be understood that read sequences are not necessary in all cases. In some embodiments, the nucleic acid probe may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences. The read sequences may be positioned anywhere within the nucleic acid probe. If more than one read sequence is present, the read sequences may be positioned next to each other, and/or interspersed with other sequences.

The read sequences, if present, may be of any length. If more than one read sequence is used, the read sequences may independently have the same or different lengths. For instance, the read sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The read sequence may be arbitrary or random in some embodiments. In certain cases, the read sequences are chosen so as to reduce or minimize homology with other components of the cell or other sample, e.g., such that the read sequences do not themselves bind to or hybridize with other nucleic acids suspected of being within the cell or other sample. In some cases, the homology may be less than 10%, less than 8%, less than 7%, less than 6%, less than 5%, less than 4%, less than 3%, less than 2%, or less than 1%. In some cases, there may be a homology of less than 20 basepairs, less than 18 basepairs, less than 15 basepairs, less than 14 basepairs, less than 13 basepairs, less than 12 basepairs, less than 11 basepairs, or less than 10 basepairs. In some cases, the basepairs are sequential.

In one set of embodiments, a population of nucleic acid probes may contain a certain number of read sequences, which may be less than the number of targets of the nucleic acid probes in some cases. Those of ordinary skill in the art will be aware that if there is one signaling entity and n read sequences, then in general 2″-1 different nucleic acid targets may be uniquely identified. However, not all possible combinations need be used. For instance, a population of nucleic acid probes may target 12 different nucleic acid sequences, yet contain no more than 8 read sequences. As another example, a population of nucleic acids may target 140 different nucleic acid species, yet contain no more than 16 read sequences. Different nucleic acid sequence targets may be separately identified by using different combinations of read sequences within each probe. For instance, each probe may contain 1, 2, 3, 4, 5, 6, 7, 8, 9, 10, 11, 12, 13, 14, 15, etc. or more read sequences. In some cases, a population of nucleic acid probes may each contain the same number of read sequences, although in other cases, there may be different numbers of read sequences present on the various probes.

As a non-limiting example, a first nucleic acid probe may contain a first target sequence, a first read sequence, and a second read sequence, while a second, different nucleic acid probe may contain a second target sequence, the same first read sequence, but a third read sequence instead of the second read sequence. Such probes may thereby be distinguished by determining the various read sequences present or associated with a given probe or location, as discussed herein.

In addition, the nucleic acid probes (and their corresponding, complimentary sites on the encoding probes), in certain embodiments, may be made using only 2 or only 3 of the 4 bases, such as leaving out all the “G”s or leaving out all of the “C”s within the probe. Sequences lacking either “G”s or “C”s may form very little secondary structure in certain embodiments, and can contribute to more uniform, faster hybridization.

In some embodiments, the nucleic acid probe may contain a signaling entity. It should be understood that signaling entities are not required in all cases, however; for instance, the nucleic acid probe may be determined using secondary nucleic acid probes in some embodiments, as is discussed in additional detail below.

Other components may also be present within a nucleic acid probe as well. For example, in one set of embodiments, one or more primer sequences may be present, e.g., to allow for enzymatic amplification of probes. Those of ordinary skill in the art will be aware of primer sequences suitable for applications such as amplification (e.g., using PCR or other suitable techniques). Many such primer sequences are available commercially. Other examples of sequences that may be present within a primary nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally-occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In addition, the components of the nucleic acid probe may be arranged in any suitable order. For instance, in one embodiment, the components may be arranged in a nucleic acid probe as: primer-read sequences-targeting sequence-read sequences-reverse primer. The “read sequences” in this structure may each contain any number (including 0) of read sequences, so long as at least one read sequence is present in the probe. Non-limiting example structures include primer-targeting sequence-read sequences-reverse primer, primer-read sequences-targeting sequence-reverse primer, targeting sequence-primer-targeting sequence-read sequences-reverse primer, targeting sequence-primer-read sequences-targeting sequence-reverse primer, primer-target sequence-read sequences-targeting sequence-reverse primer, targeting sequence-primer-read sequence-reverse primer, targeting sequence-read sequence-primer, read sequence-targeting sequence-primer, read sequence-primer-targeting sequence-reverse primer, etc. In addition, the reverse primer is optional in some embodiments, including in all of the above-described examples.

After introduction of the nucleic acid probes into a cell or other sample, the nucleic acid probes may be directly determined by determining signaling entities (if present), and/or the nucleic acid probes may be determined by using one or more secondary nucleic acid probes, in accordance with certain embodiments. As mentioned, in some cases, the determination may be spatial, e.g., in two or three dimensions. In addition, in some cases, the determination may be quantitative, e.g., the amount or concentration of a primary nucleic acid probe (and of a target nucleic acid) may be determined. Additionally, the secondary probes may comprise any of a variety of entities able to hybridize a nucleic acid, e.g., DNA, RNA, LNA, and/or PNA, etc., depending on the application.

A secondary nucleic acid probe may contain a recognition sequence able to bind to or hybridize with a read sequence of a primary nucleic acid probe. In some cases, the binding is specific, or the binding may be such that a recognition sequence preferentially binds to or hybridizes with only one of the read sequences that are present. The secondary nucleic acid probe may also contain one or more signaling entities. If more than one secondary nucleic acid probe is used, the signaling entities may be the same or different.

The recognition sequences may be of any length, and multiple recognition sequences may be of the same or different lengths. If more than one recognition sequence is used, the recognition sequences may independently have the same or different lengths. For instance, the recognition sequence may be at least 5, at least 10, at least 15, at least 20, at least 25, at least 30, at least 35, at least 40, or at least 50 nucleotides in length. In some cases, the recognition sequence may be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30, between 20 and 40, or between 25 and 35 nucleotides, etc. In one embodiment, the recognition sequence is of the same length as the read sequence. In addition, in some cases, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to a read sequence of the primary nucleic acid probe.

As discussed, in certain embodiments, nucleic acid probes are used that contain various “read sequences.” For example, a population of primary nucleic acid probes may contain certain “read sequences” which can bind certain of the secondary nucleic acid probes, and the locations of the primary nucleic acid probes are determined within the sample using secondary nucleic acid probes, e.g., which comprise a signaling entity. As mentioned, in some cases, a population of read sequences may be combined in various combinations to produce different nucleic acid probes, e.g., such that a relatively small number of read sequences may be used to produce a relatively large number of different nucleic acid probes.

Thus, in some cases, a population of primary nucleic acid probes (or other nucleic acid probes) may each contain a certain number of read sequences, some of which are shared between different primary nucleic acid probes such that the total population of primary nucleic acid probes may contain a certain number of read sequences. A population of nucleic acid probes may have any suitable number of read sequences. For example, a population of primary nucleic acid probes may have 1, 2, 3, 4, 5, 6, 7, 8, 9, 10, 11, 12, 13, 14, 15, 16, 17, 18, 19, 20 etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible read sequences present, although some or all of the probes may each contain more than one read sequence, as discussed herein. In addition, in some embodiments, the population of nucleic acid probes may have no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, or no more than two read sequences present. Combinations of any of these are also possible, e.g., a population of nucleic acid probes may comprise between 10 and 15 read sequences in total.

As a non-limiting example of an approach to combinatorially producing a relatively large number of nucleic acid probes from a relatively small number of read sequences, in a population of 6 different types of nucleic acid probes, each comprising one or more read sequences, the total number of read sequences within the population may be no greater than 4. It should be understood that although 4 read sequences are used in this example for ease of explanation, in other embodiments, larger numbers of nucleic acid probes may be realized, for example, using 5, 8, 10, 16, 32, etc. or more read sequences, or any other suitable number of read sequences described herein, depending on the application. Referring now to FIG. 13A, if each of the primary nucleic acid probes contains two different read sequences, then by using 4 such read sequences (A, B, C, and D), up to 6 probes may be separately identified. It should be noted that in this example, the ordering of read sequences on a nucleic acid probe is not essential, i.e., “AB” and “BA” may be treated as being synonymous (although in other embodiments, the ordering of read sequences may be essential and “AB” and “BA” may not necessarily be synonymous). Similarly, if 5 read sequences are used (A, B, C, D, and E) in the population of primary nucleic acid probes, up to 10 probes may be separately identified, as is shown in FIG. 13B. For example, one of ordinary skill in the art would understand that,

$$( \begin{matrix}n \\ k\end{matrix} )$$

different for k read sequences in a population with n read sequences on each probe, up to probes may be produced, assuming that the ordering of read sequences is not essential; because not all of the probes need to have the same number of read sequences and not all combinations of read sequences need to be used in every embodiment, either more or less than this number of different probes may also be used in certain embodiments. In addition, it should also be understood that the number of read sequences on each probe need not be identical in some embodiments. For instance example, some probes may contain 2 read sequences while other probes may contain 3 read sequences.

In some embodiments, the read sequences and/or the pattern of binding of nucleic acid probes within a sample may be used to define an error-detecting and/or an error-correcting code, for example, to reduce or prevent misidentification or errors of the nucleic acids. Thus, for example, if binding is indicated (e.g., as determined using a signaling entity), then the location may be identified with a “1”; conversely, if no binding is indicated, then the location may be identified with a “0” (or vice versa, in some cases). Multiple rounds of binding determinations, e.g., using different nucleic acid probes, can then be used to create a “codeword,” e.g., for that spatial location. In some embodiments, the codeword may be subjected to error detection and/or correction. For instance, the codewords may be organized such that, if no match is found for a given set of read sequences or binding pattern of nucleic acid probes, then the match may be identified as an error, and optionally, error correction may be applied sequences to determine the correct target for the nucleic acid probes. In some cases, the codewords may have fewer “letters” or positions that the total number of nucleic acids encoded by the codewords, e.g. where each codeword encodes a different nucleic acid.

Such error-detecting and/or the error-correction code may take a variety of forms. A variety of such codes have previously been developed in other contexts such as the telecommunications industry, such as Golay codes or Hamming codes. In one set of embodiments, the read sequences or binding patterns of the nucleic acid probes are assigned such that not every possible combination is assigned.

For example, if 4 read sequences are possible and a primary nucleic acid probe contains 2 read sequences, then up to 6 primary nucleic acid probes could be identified; but the number of primary nucleic acid probes used may be less than 6. Similarly, for k read sequences in a population with n read sequences on each primary nucleic acid probe,

$$( \begin{matrix}n \\ k\end{matrix} )$$

different probes may be produced, but the number of primary nucleic acid probes that are used may be any number more or less than

$$( \begin{matrix}n \\ k\end{matrix} ) .$$

In addition, these may be randomly assigned, or assigned in specific ways to increase the ability to detect and/or correct errors.

As another example, if multiple rounds of nucleic acid probes are used, the number of rounds may be arbitrarily chosen. If in each round, each target can give two possible outcomes, such as being detected or not being detected, up to 2″ different targets may be possible for n rounds of probes, but the number of nucleic acid targets that are actually used may be any number less than 2″. For example, if in each round, each target can give more than two possible outcomes, such as being detected in different color channels, more than 2″ (e.g. 3″, 4″ . . . ) different targets may be possible for n rounds of probes. In some cases, the number of nucleic acid targets that are actually used may be any number less than this number. In addition, these may be randomly assigned, or assigned in specific ways to increase the ability to detect and/or correct errors.

For example, in one set of embodiments, the codewords or nucleic acid probes may be assigned within a code space such that the assignments are separated by a Hamming distance, which measures the number of incorrect “reads” in a given pattern that cause the nucleic acid probe to be misinterpreted as a different valid nucleic acid probe. In certain cases, the Hamming distance may be at least 2, at least 3, at least 4, at least 5, at least 6, or the like. In addition, in one set of embodiments, the assignments may be formed as a Hamming code, for instance, a Hamming (7, 4) code, a Hamming (15, 11) code, a Hamming (31, 26) code, a Hamming (63, 57) code, a Hamming (127, 120) code, etc. In another set of embodiments, the assignments may form a SECDED code, e.g., a SECDED (8,4) code, a SECDED (16,4) code, a SCEDED (16, 11) code, a SCEDED (22, 16) code, a SCEDED (39, 32) code, a SCEDED (72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, a code with the same error correcting properties of the SECDED code may be formed by using only binary words that contain a fixed number of ‘1’ bits, such as 4, to encode the targets. In another set of embodiments, the assignments may represent a subset of the possible values taken from codes described above for the purpose of addressing asymmetric readout errors. For example, in some cases, a code in which the number of ‘1’ bits may be fixed for all used binary words may eliminate the biased measurement of words with different numbers of ‘1’s when the rate at which ‘0’ bits are measured as ‘1’s or ‘1’ bits are measured as ‘0’s are different.

Accordingly, in some embodiments, once the codeword is determined (e.g., as discussed herein), the codeword may be compared to the known nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc.

As discussed above, in certain aspects, signaling entities are determined, e.g., to determine nucleic acid probes and/or to create codewords. In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of entities within the sample may be determined in two or even three dimensions. In addition, in some embodiments, more than one signaling entity may be determined at a time (e.g., signaling entities with different colors or emissions), and/or sequentially.

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities optically, e.g., using fluorescence microscopy. In some cases, the spatial positions may be determined at super resolutions, or at resolutions better than the wavelength of light or the diffraction limit. Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM

(Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), or the like.

The following documents are incorporated herein by reference: U.S. Pat. Nos. 10,240,146, 11,098,303; US Pat. Apl. Pub. Nos. 2017/0212986, 2017/0220733, 2019/0233812, 2019/0264270, 2019/0276881, 2022/0025442, 2022/0064697, and 2022/0205983; and Int. Pat. Apl. Pub. Nos. WO 2016/018960, WO 2016/018963, WO 2018/089438, WO 2018/089445, WO 2018/218150, WO 2020/123742, WO 2020/214885, WO 2021/102122, and WO 2021/138078.

In addition, U.S. Provisional Patent Application Ser. No. 63/506,283, filed Jun. 5, 2023, entitled “Three-Dimensional Single-Cell Transcriptome Imaging of Thick Tissues,” by Zhuang, et al., is incorporated herein by reference in its entirety.

The following examples are intended to illustrate certain embodiments of the present disclosure, but do not exemplify the full scope of the disclosure.

### Example 1

This example presents a method to allow three-dimensional (3D) imaging of thick tissue specimens by combination of confocal microscopy for optical sectioning and deep learning for increasing imaging speed and quality. This example also presents a method to allow three-dimensional (3D) single-cell genome-scale imaging of nucleic acids such as RNA, DNA, and/or epigenetic elements in thick tissue specimens by integrating MERFISH with confocal microscopy and deep learning. This example demonstrates 3D MERFISH on mouse brain tissue sections of up to 200 micrometer thickness with high detection efficiency and accuracy. It is expected that 3D MERFISH imaging of thick tissues, and 3D thick-tissue imaging in general, will facilitate a wide range of biological applications.

The approach in this example addresses the above-described challenges by using spinning disk confocal microscopy to eliminate out-of-focus fluorescence background, exploring deep learning to speed up confocal imaging process, utilizing an index-matched objective to remove depth-induced spherical aberration, and optimizing the MERFISH protocol for thick-tissue imaging. Although this example presents a demonstration for determining RNA species in thick tissue samples, it should be understood that the present disclosure is not so limited, and this is generalizable, for example, to measure protein and/or other nucleic acids such as DNA and epigenetic elements, e.g., as discussed herein.

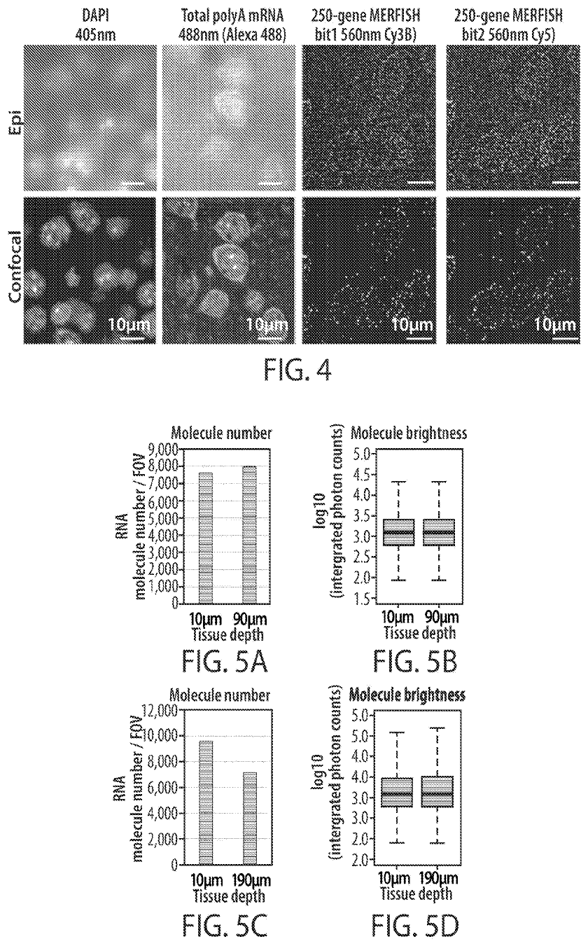

This example uses spinning disk confocal microscopy to achieve optical sectioning and eliminate the out-of-focus fluorescence background, which significantly improved the signal-to-noise ratio (SNR) in the MERFISH images of thick tissue sections (FIG. 4). However, the spinning-disk confocal detection geometry also cuts a large amount of in-focus fluorescence signals, and hence a substantially longer exposure time per imaging frame or higher illumination light intensity is required to achieve a high SNR for imaging individual RNA molecules in the sample. This led to either a drastic reduction in imaging speed or a substantial photobleaching of out-of-focus fluorophores before they were imaged.

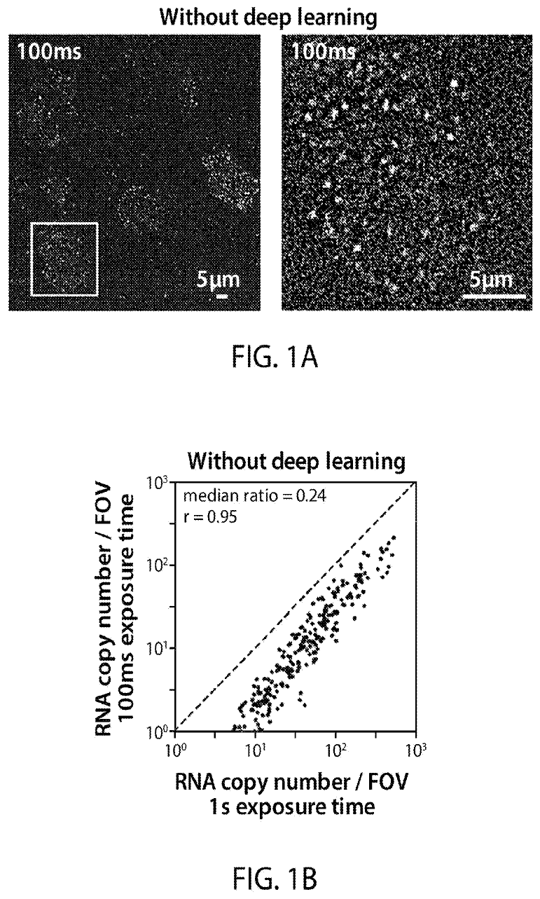

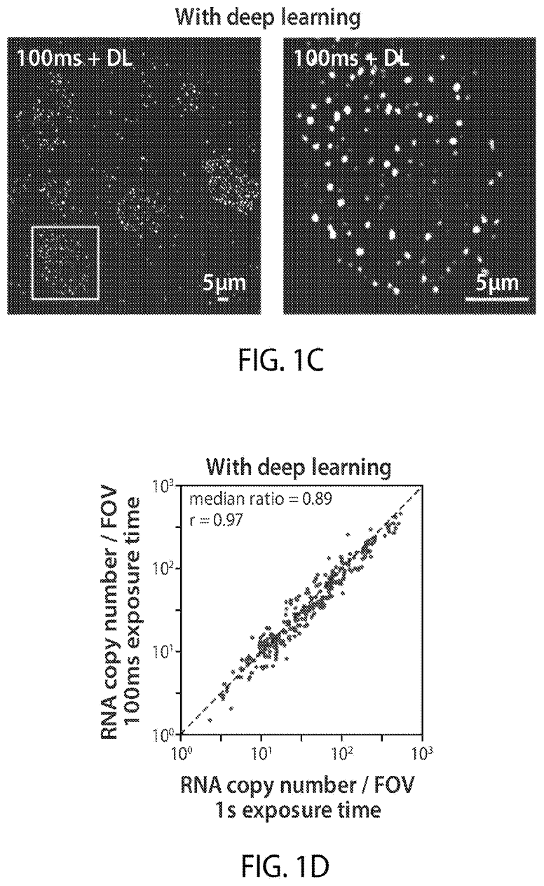

Deep learning, which has been used to improve the quality of fluorescence microscopy images in a variety of applications, could potentially improve the SNR of confocal MERFISH images acquired at high speed or with low illumination intensity. To test this approach, MERFISH was performed to measure 242 genes in the mouse cortex sections, imaging the same fields of view (FOVs) with both low (1 sec) and high (0.1 sec) frame rates to obtain high and low SNR image pairs, respectively. As expected, the low-SNR MERFISH images acquired at a frame rate of 0.1-see led to a substantially lower (4× lower) detection efficiency compared to the high-SNR measurement acquired at the 1-see exposure time (FIGS. 1A and 1B).

To test whether deep learning can enhance the quality of the 0.1-see images, a neural network was trained based on a subset of the short- and long-exposure-time image pairs and subsequently used this model to improve the quality of the remaining images taken with short exposure time. This deep-learning approach indeed improved the SNR of the 0.1-see images substantially (FIG. 1C). As a result, the detection accuracy of MERFISH images acquired at 0.1-see frame rate were increased and became nearly identical to that measured at 1-see frame rate (FIG. 1D). This advance allowed the acquisition of high-quality confocal MERFISH images at fast speed under low illumination intensity.

In thick-tissue imaging, it can be important in some cases to avoid aberration caused by refractive index mismatch. High numerical-aperture (NA=1.4) oil-immersion objectives have been previously used for thin-tissue MERFISH imaging. While these objectives have high detection efficiency of fluorescence signals from thin samples closed to glass substrate, they present significant challenges when imaging thick samples. The discrepancy in the refractive index between the immersion oil and tissue samples leads to substantial aberration which reduces both the sensitivity and accuracy of RNA detection in the deeper regions of thick samples. To overcome this problem, water-immersion objectives were used, which have a better refractive-index match with the samples. Despite having a lower NA (NA=1.15), these water objectives allowed detection of individual RNA molecules without noticeable decay of detection sensitivity and efficiency across the entire depth of 100-micrometer and 200-micrometer thick tissue sections (FIGS. 5A-D).

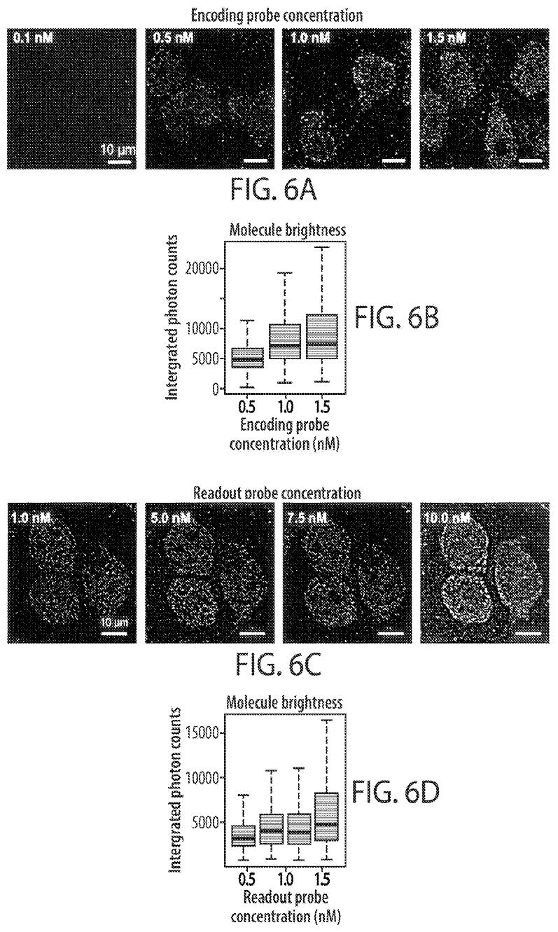

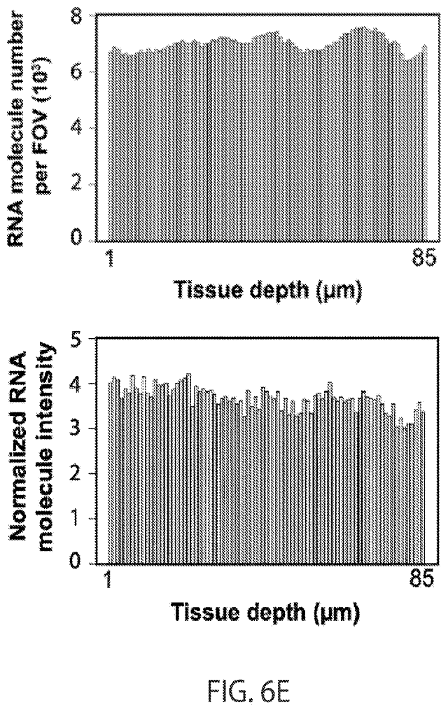

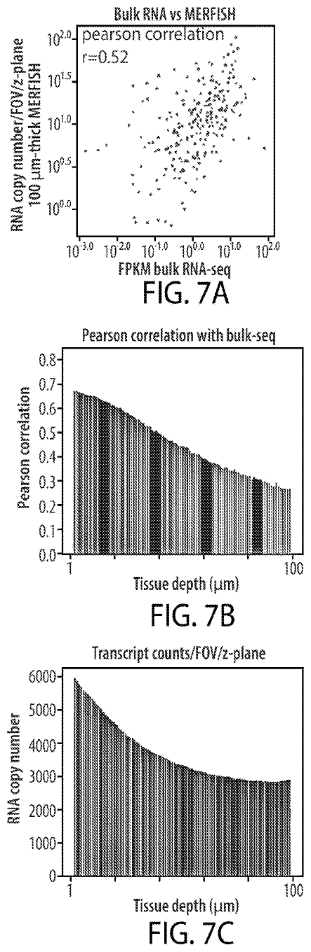

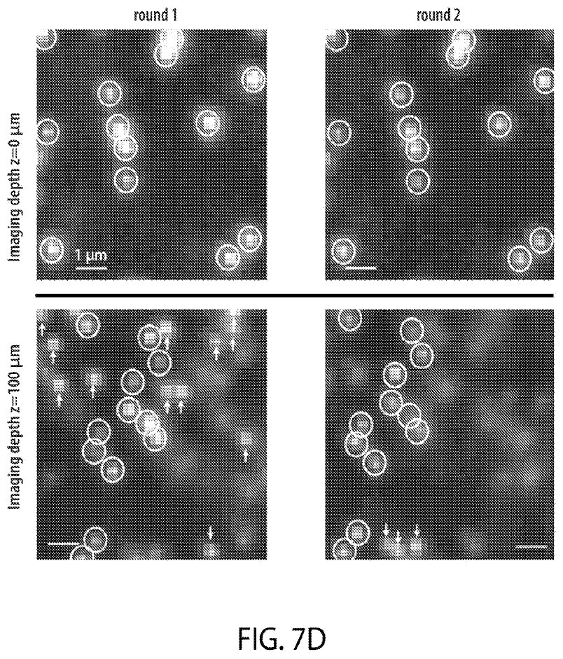

For thick-tissue optimization, 242 genes were imaged in 100-micrometer thick mouse brain sections with 1-micrometer step size for each z plane. In MERFISH, cellular RNAs were labeled with a library of encoding oligonucleotide probes which contains barcode-determining readout sequences and then the barcodes were detected bit-by-bit with readout oligonucleotide probes conjugated with fluorophores. Upon optimization of probe concentrations and incubation time (FIGS. 6A-6E), bright and consistent signals from individual RNA molecules were observed across the entire depth of 100-micrometer thick tissue samples in each bit. Unexpectedly, despite consistent detection of RNA molecules in individual bits, the RNA copy number detected for individual genes per z-plane decreased substantially with the tissue depth and exhibited a poor correlation with bulk RNA-seq data (FIGS. 7A-7C). This may be due to the displacement of RNA molecules between imaging rounds in the thick tissue sample which made these molecules difficult to decode and identify from their multi-bit images. To test this, fiducial beads embedded in the polyacrylamide gel were imaged, which is used for MERFISH sample embedding. A significant displacement in the positions of beads in all three dimensions (xyz) was observed between imaging rounds, especially in the deeper part of the sample (FIG. 7D).

The displacement of RNA molecule positions between imaging rounds could be attributed to one or more factors. For example, the piezo-actuator used for z-scanning may not have consistently placed the sample at the pre-defined z position during each imaging round. The polyacrylamide gel, prone to expansion or shrinkage when buffer conditions alter, may have contributed to the displacement of RNA molecules across imaging rounds due to its inconsistent size changes. The two-color imaging was used to measure two bits in each hybridization round, and the axial chromatic aberration between the two colors, which increases with imaging depth, may have resulted in misalignment of RNA molecules between bits.

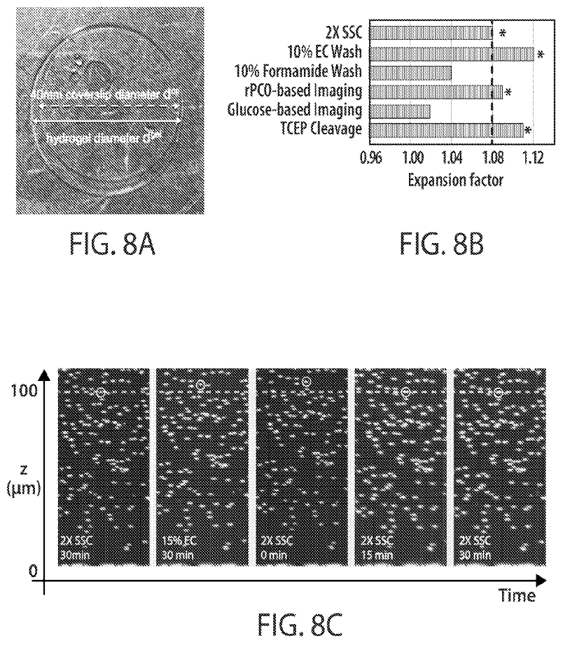

To solve the first problem, different piezo actuators were tested, and the piezo actuator with the highest precision and reproducibility (Queensgate OP400) was selected for these experiments, which provided negligible z-position error for sample thicknesses under 200 micrometers. To minimize the gel expansion effect, a low concentration of gel initiator and low polymerization temperature (4° C.) was used to generate polyacrylamide gel with a lower degree of buffer-dependent expansion, and MERFISH buffers were chosen that further minimize gel expansion effect (FIGS. 8A-8B). In addition, to ensure that the gel recovered to the same size before imaging, the gel was allowed to relax for 10 minutes following two rounds of washing using the imaging buffer in order to completely remove any residual cleavage or probe-incubation buffer induced gel expansion (FIG. 8C). To eliminate the effect of chromatic aberration, the axial chromatic aberration was calibrated by imaging fiducial beads in the two-color channels, which allowed precise alignment of images between these channels.

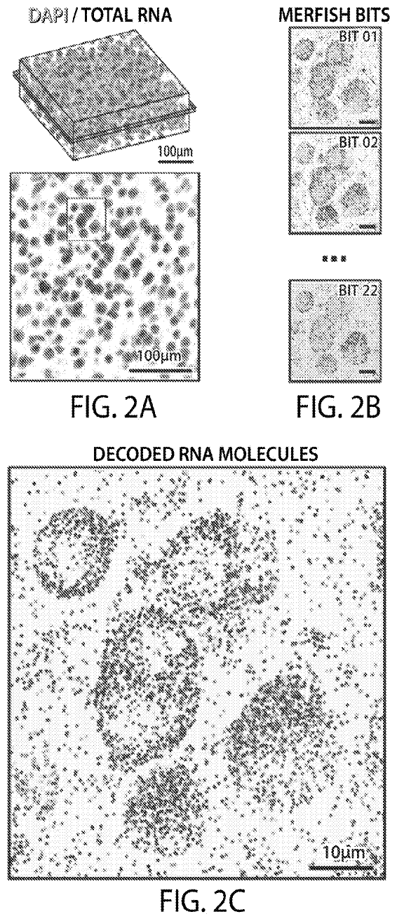

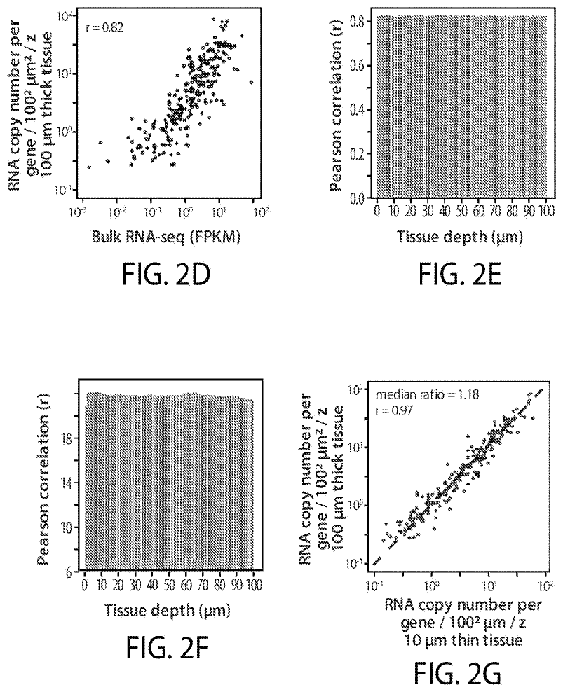

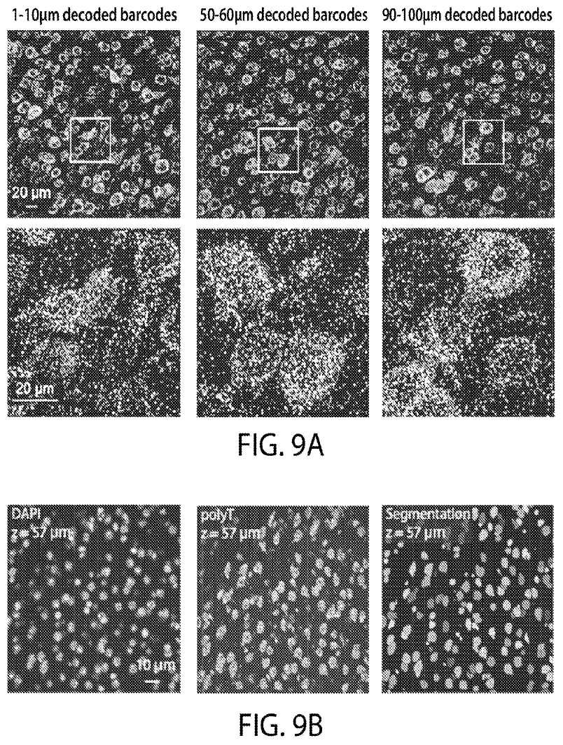

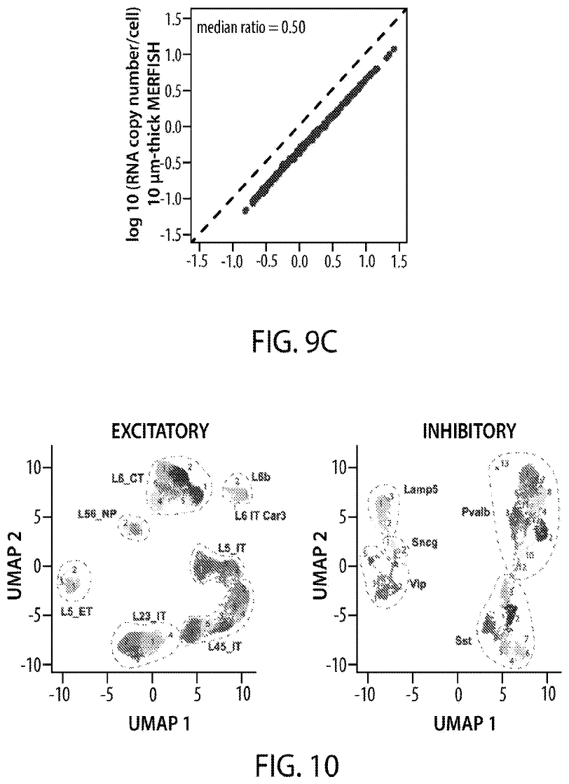

Next, the optimized thick-tissue MERFISH protocol was validated by first imaging the expression of 242 genes in 100-micrometer thick mouse cortex sections. In addition to MERFSH imaging of RNAs, the DAPI stain and total polyadenylated mRNA signals for 3D cell segmentation was imaged (FIG. 2A). Individual RNA molecules were identified in the thick-tissue MERFISH images (FIGS. 2B-2C; FIG. 9A). The average RNA copy numbers for individual genes were highly correlated with the RNA abundance measured by bulk RNA sequencing (Pearson correlation coefficient r=0.82) (FIG. 2D). The detected transcript number and the correlation with bulk RNA-seq did not show any decay across the entire tissue depth (FIG. 2e, f). The RNA copy number of individual genes detected per unit area per z-plane were compared with the results from 10 micrometers thin-tissue MERFISH measurements performed previously using an epi-fluorescence setup. The detection efficiency of thick-tissue measurements was ~20% higher than the thin-tissue measurements (FIG. 2G) which may benefit from the background reduction by confocal optical sectioning. Given that confocal imaging provides reduced depth per z-sectioning compared to epi-fluorescence imaging, the detection efficiency of thick-tissue measurements by 3D MERFISH could potentially be even higher.

3D cell segmentation was performed using DAPI and total polyadenylated mRNA signals (FIG. 9B), which allowed the determination of the expression profiles of the 242 genes in each individual cell. When the RNA copy number per cell detected in the 100-micrometer thick samples was compared with the results from individual 10-micrometer thick sections of the same sample (obtained by dividing the 100-micrometer z-range into 10 equal-thickness sections), it was found that the former was two-fold higher than the latter (FIG. 9C). This is believed to be because most cell bodies were completely captured within the 100 micrometer-thick tissues, whereas many cells were only partially captured in the 10 micrometer-thick tissue sections. Although normalization of the RNA copy number by cell volume could partially mitigate this problem in thin-tissue imaging, detecting too few RNA molecules per cell could nonetheless induce significant noise and impose a cell volume threshold to reduce noise would in turn reduce the number of cells measured. Moreover, non-uniform intracellular distribution of RNAs could further reduce transcriptome profiling accuracy when only part of a cell is imaged. Thus, thick-tissue MERFISH imaging should allow more accurate gene expression profiling of whole cells.

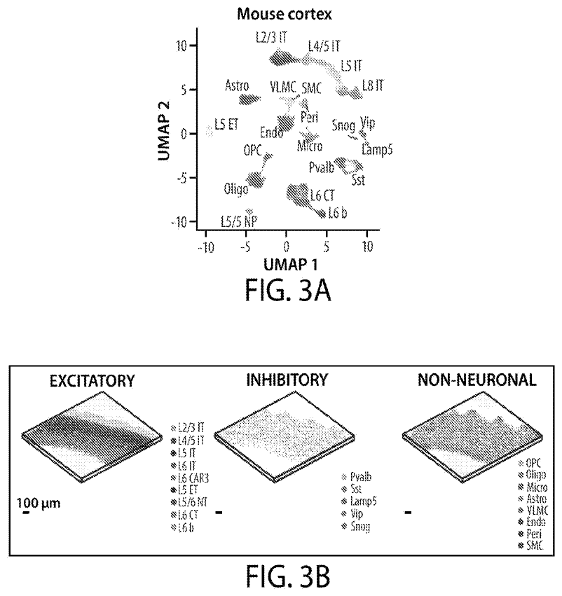

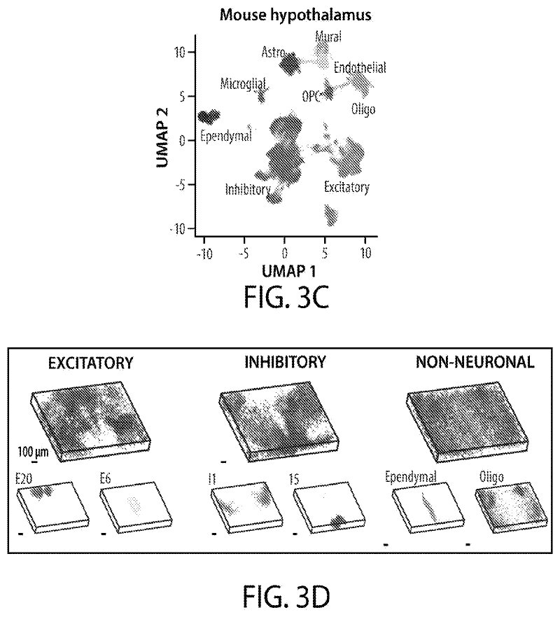

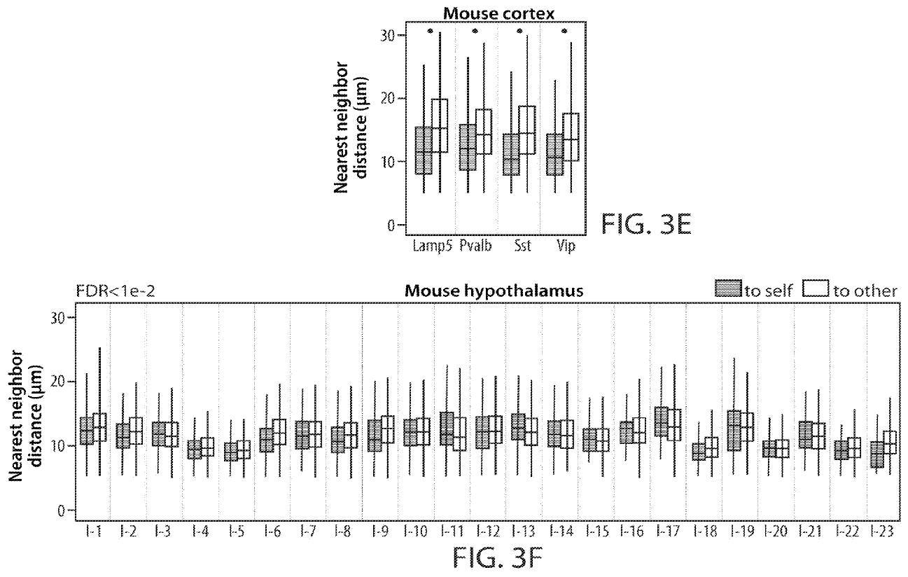

Next, single-cell expression profiles derived from the 3D MERFISH measurements were used to identify transcriptionally distinct cell populations in the mouse cortex. All previously known subclasses of excitatory neurons (L2/3 IT, L4/5 IT, L5 IT, L6 IT, L5 ET, L5/6 NP, L6 CT and L6b), inhibitory neurons (marked by Lamp5, Sst, Vip, Pvalb and Sncg respectively), non-neuronal cells (oligodendrocytes, oligodendrocyte progenitor cells, astrocytes, microglia, endothelial cells, pericytes, VLMC, smooth muscle cells) (FIG. 3A) as well as transcriptionally distinct cell clusters within these subclasses (FIG. 10) were observed. The 3D MERFISH images further showed the expected layered organization of transcriptomically distinct neuronal cell populations in the cortex (FIG. 3B).

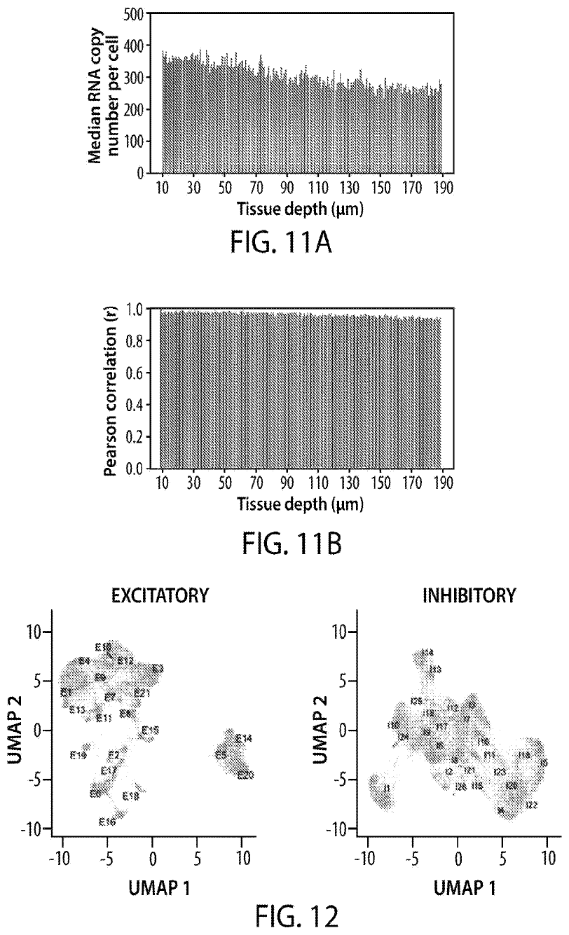

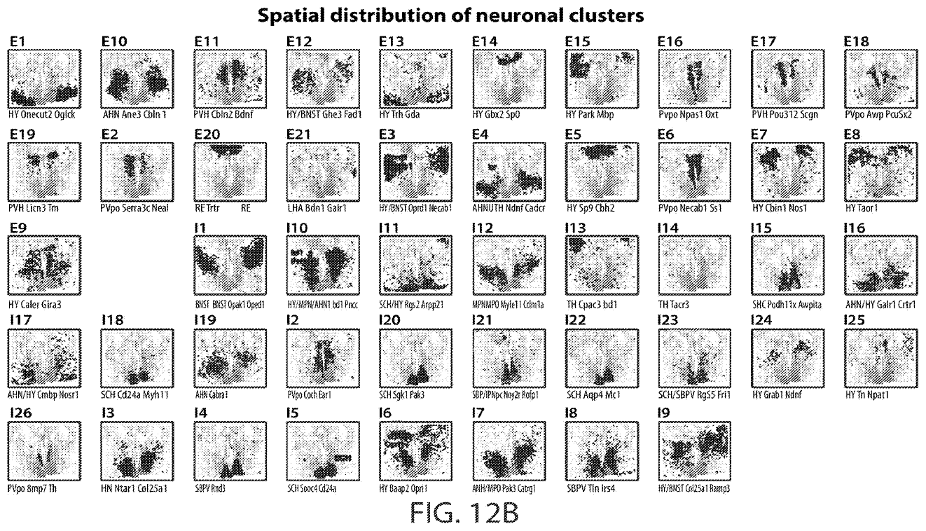

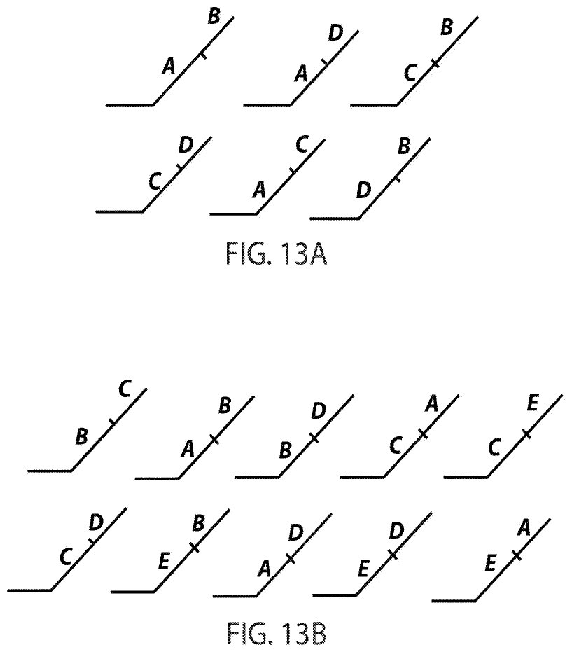

To test if the thick-tissue 3D-MERFISH approach could image beyond 100-micrometer thickness, next, 156 genes in a 200-micrometer thick mouse anterior hypothalamus section were measured. The RNA copy numbers of individual genes measured per cell per z-plane at different tissue depths showed excellent correlation with each other with only a slight reduction of the RNA copy number across the entire tissue depth (FIGS. 11A, 11B). From the MERFISH-derived single-cell expression profiles, 21 excitatory neuronal clusters, 26 inhibitory neuronal clusters, and 7 non-neuronal cell subclasses were identified in this region (FIG. 3C, 3D; FIG. 12A). Most of the transcriptionally distinct neuronal clusters showed distinct and localized spatial distributions, some of which were predominantly located in a single hypothalamic nucleus such as cluster Il in the bed nucleus of the stria terminalis (BNST), cluster 15 in the suprachiasmatic nucleus (SCH), and cluster E20 in the reuniens thalamic nucleus (RE) (FIG. 3D; FIG. 12B).

It has been shown previously that interneurons (Lamp5, Vip, Sst, Pvalb positive inhibitory neurons) in the mouse and human cortex tend to form juxtaposed structures with another cell in the same type, as reflected by the observations that the nearest-neighboring distance between the same type of interneurons are significantly smaller than that between this type of inhibitory neurons and other cell types, despite the fact that the density of interneurons in the cortex is much lower than that of the excitatory neurons. This was confirmed with analysis of the thick-tissue MERFISH data (FIG. 3E, left), as well as whole-brain MERFISH data that covers multiple cortical regions in the mouse brain (FIG. 3E, right). It has been hypothesized that such juxtaposed structures might be mediated by electric gap junctions which is important for synchronized firing patterns. Whether such juxtaposed inhibitory neuronal pairs were also formed in the hypothalamus were tested by performing the same nearest neighbor analysis. Interestingly, unlike inhibitory neurons in the cortex, inhibitory neurons in the anterior hypothalamus did not show preferential juxtaposition with inhibitory neurons of the same type (FIG. 3F).

In summary, this example demonstrates a method that allows high-performance 3D MERFISH imaging of thick tissue samples. This method will benefit many important applications. First, as shown here, imaging thick tissues allows the capture of the full volume of nearly all cells, and hence increases the transcriptome profiling accuracy. Second, confocal imaging removes out-of-focus fluorescence background, which can potentially allow the imaging of more genes or the same number of genes with fewer imaging rounds. Third, confocal imaging can be used to facilitate cell segmentation, especially in regions with high cell density. Fourth, thick-tissue MERFISH imaging should facilitate integrated measurements of gene expression profiles and morphology of individual cells to investigate the relationship between these two properties. Finally, this approach may also facilitate combined measurements of neuronal activity, for example by calcium imaging, with transcriptome profiles of individual cells to reveal the functional roles of molecularly defined cell types.

FIG. 1. Deep learning enhances performance of confocal MERFISH imaging. (FIG. 1A). A single-bit 242-gene high-pass-filtered MERFISH confocal image in a brain tissue section taken with an exposure time of 0.1 sec (left) and a magnified view of a single cell marked by the white box in the left image for a closer examination (right). (FIG. 1B) The correlation between the copy number of individual genes detected per field-of-view (FOV) using 0.1-see frame rate and those obtained using 1-see frame rate. The median ratio of the copy number and the Pearson correlation coefficient r are shown. (FIG. 1C) The same image as in (FIG. 1A) but after enhancement of signal-to-noise ratio (SNR) by a deep-learning algorithm. (FIG. 1D) The same as (FIG. 1B) but after deep learning was used to enhance the SNR of the 0.1-see images.

FIG. 2. 3D-MERFISH imaging of thick brain tissue sections. (FIG. 2A) 3D images of DAPI and total polyA mRNA from a single FOV in a 100-micrometer thick mouse brain tissue slice (top), alongside a single z-plane at tissue depth of 50 micrometer marked by the box in the top image (bottom). (FIG. 2B) Maximum projected high-pass-filtered MERFISH bit images of ten consecutive 1-micrometer z-planes, captured for the cells marked in box in FIG. 2A, bottom. (FIG. 2C) RNA molecules identified in the same region as (FIG. 2B) with RNA molecules shaded by their genetic identities. (FIG. 2D) The RNA copy number for individual genes per unit area (100² micrometers²) per z-plane detected in the 100-micrometer MERFISH measurements of mouse cortex versus the FPKM from bulk RNA-seq. r stands for the Pearson correlation coefficient. (FIG. 2E) The Pearson correlation between RNA copy number for individual genes per z-plane detected by MERFISH and FPKM from bulk RNA-seq at different tissue depths. (FIG. 2F) Number of detected RNA molecules per FOV at different tissue depths. (FIG. 2G) The RNA copy number for individual genes per unit area (100² micrometer²) per z-plane detected in the 100-micrometer MERFISH measurements of mouse brain sections versus that detected by 10-μm thin-tissue MERFISH measurements by an epifluorescence setup. r stands for the Pearson correlation coefficient.

FIG. 3. Spatial organization of cell types in the mouse cortex and hypothalamus by 3D thick tissue MERFISH. (FIG. 3A) UMAP visualization of subclasses of cells identified in a 100 micrometer-thick section in the mouse cortex. Cells are shaded by subclass identities. (FIG. 3B) 3D spatial maps of the identified subclasses of excitatory neurons (left), inhibitory neurons (middle) and non-neuronal cells (right) within the 100-micrometer mouse cortex-section. (FIG. 3C) UMAP visualization of major cell types identified in a 200 micrometer-thick section of the mouse anterior hypothalamus. Cells are shaded by cell type identities. (FIG. 3D) 3D spatial maps of the excitatory neuronal (left) and inhibitory neuronal (middle) and non-neuronal cells (right) identified in the 200 micrometer-thick mouse hypothalamus section. (FIG. 3E) Distributions of the nearest-neighbor distances from cells in individual inhibitory neuronal subclasses to cells in the same subclass (“to self”) or other subclasses (“to other”) in mouse cortices measured by MERFISH in the mouse cortex. (FIG. 3F) Distributions of the nearest-neighbor distances in the mouse anterior hypothalamus as descried in (FIG. 3E). * FDR <0.01 in (FIG. 3E) and (FIG. 3F) was determined with the Wilcoxon rank-sum one-sided test and adjusted to FDR by the BH procedure. Only inhibitory neuronal clusters with at least 20 “self-self” interacting pairs were examined and plotted in (FIGS. 3E-3F).

FIG. 4. Comparison of epifluorescence and confocal MERFISH images in thick tissue samples. Images of nuclei, total polyA mRNA, and two MERFISH bits were obtained using epifluorescence and confocal microscopy respectively. Both confocal and epifluorescence images were taken with 1-see exposure time. The MERFISH bits images were high-pass filtered to remove cellular background.

FIG. 5. MERFISH images of RNA molecules at different tissue depths in 100-micrometer and 200-micrometer thick brain tissue sections. (FIG. 5A) Number of RNA molecules detected per FOV at the tissue depths of 10 micrometers and 90 micrometers of the 242-gene MERFISH measurements in a 100-micrometer thick section of the mouse cortex. (FIG. 5B) Logarithmic distribution of integrated photon counts of individual RNA molecules at the tissue depths of 10 micrometers and 90 micrometers identified in (FIG. 5A). (FIG. 5C) Number of RNA molecules detected per FOV at tissue depths of 10 micrometers and 190 micrometers of the 156-gene MERFISH measurements in a 200-micrometer thick section of the mouse hypothalamus. (FIG. 5D) Logarithmic distribution of integrated photon counts of individual RNA molecules at the tissue depths of 10 micrometers and 190 micrometers identified in (FIG. 5C).

FIG. 6. Optimization of MERFISH encoding and readout probe labeling conditions. (FIG. 6A) Example bit-1 high-pass filtered MERFISH images of a 242-gene MERFISH measurement in a 100-micrometer thick section of mouse cortex stained with different concentrations of encoding probes. The concentration values refer to the concentration of each individual encoding probe. (FIG. 6B) Distribution of integrated photon counts of individual RNA molecules identified at different encoding probe concentrations. The signals from individual RNA molecules increased with the encoding probe concentration and reached saturation at 1.0 nM per probe. Thus, a 1 nM encoding probe concentrations for staining thick tissue samples was used. (FIG. 6C) A 100 micrometer-thick mouse brain slice was stained with the 242-gene MERFISH encoding probes, followed by sequential hybridization with readout probes corresponding to the first, second, third and fourth bit of the barcodes, each bit using a different readout probe concentration. The signal increased with readout probe concentration, but the background also increased when the probe concentration reached beyond 5 nM. A 5 nM readout probe concentration for thick-tissue imaging was thus used. In addition to the probe concentrations, the readout probe incubation time was also optimized. (FIG. 6D) The number of RNA molecules per FOV per z-plane and the normalized intensity of individual molecules at different tissue depths. The encoding probe concentration is 1 nM per encoding probe, the readout probe concentration is 5 nM, and the readout probe incubation time is 25 minutes.

FIG. 7. Displacement of RNA molecules between different imaging rounds reduces detection accuracy and efficiency. (FIG. 7A) Comparison of RNA copy number for individual genes per FOV per z-plane detected in a 242-gene MERFISH measurement of the 100 micrometer-thick mouse cortex tissue section with the FPKM values obtained by bulk RNA-seq. The Pearson correlation coefficient (r) is shown. (FIG. 7B) Pearson correlation of RNA copy number of individual genes per FOV per zplane detected at different tissue depths in the 100 micrometer-thick section with FPKM measured by bulk RNA-seq. (FIG. 7C) Total RNA copy number detected per FOV per z-plane at different tissue depths. (FIG. 7D) Example images of gel-embedded fiducial beads acquired in two rounds of imaging. Buffer exchange was performed between imaging rounds, mimicking the MERFISH protocol. Because the gel expanded to a different extent in different rounds, the positions of beads changed from rounds to rounds in xyz. Circles mark identical beads identified across two imaging rounds, while arrows highlight unique beads exclusively captured in each imaging round.

FIG. 8. Quantification of gel expansion effect by MERFISH buffers. (FIG. 8A) Quantification of gel expansion factor in various buffers used in the MERFISH protocol. The initial gel size was the same as the coverslip and the expansion factor after buffer exchange was determined as the ratio of the gel size after buffer exchange over the coverslip size. (FIG. 8B) In each round of MERFISH imaging, the sample is subjected to treatment with the readout probe within a wash buffer (either 10% ethylene carbonate EC or 10% Formamide) which is left to hybridize for a duration of 15 minutes. Subsequently, the sample was rinsed off with the wash buffer to eliminate any excessive readout probes, followed by a treatment with imaging buffer (either glucose-based or Bacterial Protocatechuate 3,4-Dioxygenase rPCO-based imaging buffer). After the imaging process, the sample is treated with Tris(2-carboxyethyl)phosphine (TCEP) cleave buffer to eliminate fluorescent signals, and finally washed with a solution of 2× Saline-Sodium Citrate (SSC) to eliminate cleavage buffer. Gel expansion factor in various buffers used for MERFISH imaging was quantified and shown here. Reagents marked by * were selected for final use in the 3D MERFISH experiment. Dash line highlights the expansion factor for 2×SSC which is the base for all other buffers, i.e. all other buffers contain 2×SSC. (FIG. 8C) XZ projection images of fiducial beads embedded in a gel undergoing buffer exchange for the indicated time period. Wash buffer containing 15% EC in 2×SSC causes noticeable gel distortion, which is recoverable after 15 min treatment of 2×SSC.

FIG. 9. 3D MERFISH imaging of 242 genes in a 100 micrometer-thick section of the mouse cortex. (FIG. 9A) Example images of decoded RNA molecules at different tissue depths. Each image shows decoded barcodes in a 10-micrometer thick z-range, as indicated. Bottom panels show the zoom-in of the region marked by white boxes in the top panels for closer examination. Identified RNA molecules are shaded by its genetic identities. (FIG. 9B) DAPI (left) and polyA mRNA (middle) images of an example field of view (FOV), which were used for cell segmentation. Cell boundary segmentation determined using a deep learning-based segmentation algorithm (Cellpose 2.0) is shown in the right panel. (FIG. 9C) The RNA copy number of individual genes per cell detected in the 100-micrometer thick tissue section versus that in individual 10-micrometer thick z-ranges of the same sample. The 100-micrometer thick section was evenly divided into 10 z-ranges to determine the latter.

FIG. 10. Cell type identified in the 100-micrometer thick section in the mouse cortex. UMAP visualization of excitatory (left) and inhibitory (right) neuronal clusters colored by their cluster identities identified in the mouse cortex.

FIG. 11. 3D MERFISH imaging of 156 genes in a 200-micrometer thick section of the mouse hypothalamus. (FIG. 11A) The median RNA copy numbers per cell along the tissue depth of the 200-micrometer thick section in the mouse hypothalamus. The first and last 10 micrometers were excluded from the analysis due to their incomplete cell coverage. (FIG. 11B) The Pearson correlation coefficient of the RNA copy numbers for individual genes along tissue depth versus those of the initial 1-micrometer of the 200-micrometer thick section.

FIG. 12. Cell type identified in the 200-micrometer thick section in the mouse hypothalamus and their spatial organization. (FIG. 12A) UMAP visualization of excitatory and inhibitory neuronal clusters colored by their cluster identities identified in the mouse anterior hypothalamus in a 200-micrometer thick section. (FIG. 12B) 2D spatial visualization of individual excitatory and inhibitory neuronal clusters. The hypothalamus nuclei in which each cluster is localized, and top 1 or 2 notable genes of each cluster are listed for individual clusters. Three clusters, specifically E20, 11, and 15, along with the corresponding nucleus in which they are localized, are marked by dashed lines.

Following are various methods and materials used in the above examples.

Animals. Adult C57BL/6J male mice aged 7-9 weeks were used in this study. Mice were maintained on a 12-h light/12-h dark cycle (12:00 noon to 12:00 midnight dark period), at a temperature of 22+/−1° C., a humidity of 30-70%, with ad libitum access to food and water. Animal care and experiments were carried out in accordance with NIH guidelines and were approved by the Harvard University Institutional Animal Care and Use Committee (IACUC).

Tissue preparation for 3D MERFISH. Mice, aged 7-9 weeks, were deeply anesthetized using isoflurane. Subsequently, a transcardial perfusion was performed with phosphate buffered saline (PBS), followed by a 4% paraformaldehyde (PFA) solution. The brain tissue was then carefully dissected and subjected to a post-fixation process in a 4% PFA solution, which was carried out overnight at 4° C. Following this, the brain tissue was thoroughly rinsed with PBS. Sections of 100 or 200-micrometer thickness were then prepared by embedding the brain tissue in 4% low melting point agarose (Thermo Fisher Scientific, 16520-050). The sections were obtained using a Vibratome (Leica). Finally, these sections were collected in 1×PBS and preserved in 70% ethanol. The sample was stored at a temperature of 4° C., and the sections were left to rest overnight for permeabilization in 70% ethanol.

The samples were removed from the 70% ethanol, washed with 2× saline sodium citrate (SSC) for three times, then equilibrated with encoding-probe wash buffer (30% formamide in 2×SSC) for 30 min at 47° C. The wash buffer was aspirated from the sample, and it was gently transferred to a 2 mL DNA low-bind centrifuge tube containing 50-microliter encoding-probe mixture. The encoding-probe mixture comprised approximately 1 nM of each encoding probe, 1 micromolar of a polyA-anchor probe (IDT), 0.1% wt/v yeast IRNA (15401-011, Life Technologies) and 10% v/v dextran sulfate (D8906, Sigma) in the encoding-probe wash buffer. The sample was incubated at 37° C. for 24-48 h. The polyAanchor probe sequence (/5Acryd/TTGAGTGGATGGAGTGTAATT+TT+TT+TT+TT+TT+TT+TT+TT+TT+T (SEQ ID NO: 1)) contained a mixture of DNA and LNA nucleotides, where T+ is locked nucleic acid, and/5Acryd/is a 5′ acrydite modification. The polyA anchor allows polyadenylated mRNAs to be anchored to the polyacrylamide gel during hydrogel embedding step as described below. After hybridization, the sample was washed three times for 20 min each at 47° C. in encoding-probe wash buffer to rinse off excessive probes, then three times in 2×SSC at room temperature.

Next the sample was embedded in a hydrogel to clear the tissue background and remove off-target probe binding. First, the sample was incubated in monomer solution with 2 M NaCl, 4% (vol/vol) of 19:1 acrylamide/bisacrylamide, 60 mM Tris-HCl pH 8, and 0.2% (vol/vol) TEMED for 30 minutes at room temperature. Then 100 microliters of ice-cold monomer solution containing 0.2% (vol/vol) 488 nm fiducial beads (Invitrogen) was placed onto a 40-mm silane coated coverslip. The silane modification procedure allowed the hydrogel to covalently couple to the coverslip surface as described previously. Next, the tissue was gently transferred to the coverslip using a brush and flattened, and the excess monomer solution was carefully aspirated. Onto a hydrophobic glass plate treated with GelSlick (Lonza), we then placed a 100 microliter droplet of the ice-cold monomer solution containing 0.1% (wt/vol) ammonium persulfate. The coverslip bearing the flattened sample was then inverted onto the droplet to form a uniform layer of monomer solution. A 50 g weight was placed on the top of the coverslip to ensure the tissue remained flat and fully attached to the coverslip. The sample was allowed to polymerize completely for a minimum of 1 hour at room temperature which allowed mobile sample slice to be fully attached to the coverslip.

The coverslip bearing the polymerized sample was then removed from the glass plate with a thin razor blade. The sample was then incubated in digestion buffer containing 2% (wt/vol) Sodium dodecyl sulfate (SDS) (ThermoFisher), 0.5% (vol/vol) Triton X-100 (ThermoFisher), and 1% (vol/vol) Proteinase K (New England Biolabs) in 2×SSC for 24 hours at 37° C. Following digestion, the sample was washed in 2×SSC buffer supplemented with 0.2% (vol/vol) Proteinase K at room temperature for 1 hour. The buffer was changed every 30 minutes to ensure sufficient washing.

MERFISH encoding and readout probes. Two sets of MERFISH encoding probes were used in this study: a MERFISH encoding probe set previously designed to target 242 genes in the mouse primary motor cortex, and another set targeting 156 genes in the hypothalamic preoptic area of the mouse brain. In previous studies of hypothalamic preoptic area, 135 out of 156 genes were imaged using combinatorial MERFISH imaging while the remaining 20 genes were measured in sequential rounds of multicolor smFISH. In this study, these 20 genes were incorporated into the combinatorial imaging rounds. Fluorescent readout probes, conjugated to either Cy5, Cy3B or Alexa488 dye molecules through a disulfide linkage were purchased from Bio-Synthesis, Inc.

3D MERFISH imaging platform. Two 3D MERFISH imaging platforms were constructed in this study. One (setup 1) was built around a Nikon Ti-U microscope body equipped with a Nikon 40×1.15 NA water immersion lens (Nikon, MRD77410) or 60×1.2 NA water immersion lens (Olympus, UPLSAPO60XW) and a spinning disk confocal unit (Andor Dragonfly, ACC-CR-DFLY-202-40). Illumination was provided with solid-state lasers at 647 nm (MBP Communications, 2RU-VFL-P-1500-647-B1R), 561 nm (MBP Communications; 2RUVFL-P-1000-560-B1R), 488 nm (Coherent, Genesis MX488-1000 STM) and 405 nm (Coherent, Obis 405-200C). The output of the 647 nm, 561 nm and 488 nm was controlled by an acousto-optic tunable filter (Crystal Technologies, AODS 20160-8 and PCAOM Vis), while the 405 nm was controlled via direct modulation. The coaligned beams were coupled into the input fiber of a beam homogenizer (Andor, Borealis BCU-120) which provided uniform illumination for the spinning disk. Fluorescence emission was isolated with a penta-bandpass dichroic (Andor, CR-DFLY-DMPN-061) and an emission filter (Andor, TR-DFLY-P45568-600) in the imaging path. Sample position was controlled by a motorized XY stage (Prior, Proscan H117EIN5/F), while z-scanning was controlled by a piezo objective nanopositioner (Queensgate, OP400 or Mad City Labs,F200S). Before image scanning, initial focus was acquired via a custom-built autofocus system that monitored the position of a reflected IR laser (Thorlabs, LP980-SF15) from the coverslip surface with a CMOS camera (Thorlabs, DCC1545M). All laser and piezo control signals were generated by a DAQ card (National Instruments, PCIe-6353) and were synced to the fire signal of the sCMOS camera (Hamamatsu, Orca-Flash4.0).

A similar imaging platform (setup 2) was built based on an Olympus IX71 microscope body, spinning disk confocal unit (Andor, CSU W1), beam homogenizer (Andor, Borealis BCU 100), piezo objective nanopositioner (Mad City Labs, Nano-F200S) and XY stage (Marzhauser, Scan IM 112×74). The illumination was provided by with solidstate lasers at 647 nm (MBP Communications, 2RU-VFL-P-2000-647-B1R), 561 nm (MPB Communications; 2RU-VFL-P-1000-560-B1R), 488 nm (MPB, 2RU-VFL-P-500-488-B1R) and 405 nm (Coherent, Cube 405), and was controlled via mechanical shutters (Uniblitz, LS6T2).

Details regarding which imaging platform was used to acquire specific data is listed in Table 1.

Fluidic system and sample chamber. MERFISH samples were imaged on 40 mm round cover glass (Bioptech) and mounted in a flow chamber (Bioptech, FCS2) using a 0.5 mm gasket (Bioptech, 1907-1422-500). The fluidic system contained a peristaltic pump (Gilson, Minipuls 3), and four eight-way valves (Hamilton, MVP 36798 with 8-5 Distribution Valve) assembled to provide up to 24 readout bit solutions, and four additional buffers (2×SSC, wash, cleave and image). Image acquisition and fluidic control were fully automated using custom-built software.

3D MERFISH imaging. To prepare the sample for imaging, it was first stained with a readout hybridization mixture containing the readout probes associated with a probe complementary to the polyA-anchor probe and conjugated via a disulfide bond to the dye Alexa488 at a concentration of 25 nM per probe. The readout hybridization mixture comprised the readout-probe wash buffer of 2×SSC, 10% v/v ethylene carbonate (E26258, Sigma) and 0.1% v/v Triton X-100, supplemented with the readout probes. The sample was incubated in this mixed buffer for 30 min at room temperature, and then washed in the readout-probe wash buffer supplemented with 1 microgram/ml DAPI for 30 min to stain nuclei within the sample. The sample was then washed in 2×SSC for 15 min loaded into the flow chamber. Imaging buffer containing 5 mM 3,4-dihydroxybenzoic acid (P5630, Sigma), 2 mM trolox (238813, Sigma), 50 micromolar trolox quinone, 1:500 recombinant protocatechuate 3,4-dioxygenase (rPCO; OYC Americas), 1:500 murine RNase inhibitor and 5 mM NaOH (to adjust pH to 7.0) in 2×SSC was introduced into the chamber. A time of at least 15 min was used to let imaging buffer fully penetrate into the deep part of the tissue. The sample was then imaged with a low-magnification objective 10× air objective using 405-nm illumination to produce a tiled imaged of the sample. This image was then used to locate the region of interest (ROI) in each slice and to generate a grid of field-of-view (FOV) positions to cover the ROI. After determining these positions, a high-numerical aperture objective was used to image each of the FOV positions. In the first round of imaging, images were collected in the 488-nm and 405-nm channels to image the 488 nm fiducial beads, the total polyA mRNA stained by the polyA-anchor probe and the nucleus stained by DAPI. These two channels were later used for cell segmentation. A single image of the fiducial beads was taken on the coverslip surface for every imaging round as a spatial reference to correct for slight differences in the stage position. 1-micrometer-thick z-stacks were collected for all channels in each FOV.

After the first round of imaging, the fluorescent dyes were removed by flowing 2 mL of cleavage buffer comprising 2×SSC and 50 mM of Tris(2-carboxyethyl)phosphine (TCEP; 646547, Sigma) with a 15-min incubation in the flow chamber in order to cleave the disulfide bond linking the dyes to the readout probes. The sample was then washed by flowing 4 mL of 2×SSC to fully remove residual cleavage buffer.

To perform subsequent rounds of imaging, 3 mL of the readout probe mixture containing the appropriate readout probes were flowed and allowed to hybridize for 25 min (15 min flow followed by a 10 min pause). Two readout probes were hybridized in each round, one labelled with Cy5 and the other with Cy3b, and a readout-probe mixture containing 5 nM of appropriate readout probes was used for each round. The sample was then washed with 2 mL of readout-probe wash buffer followed by 2 mL of imaging buffer twice for 15 min (5 min flow and 10 min pause). For each round, all FOVs were imaged in the 650-nm and 560-nm channels for the two readout probes and fiducial beads in 488-nm on the surface. The hybridization, wash, imaging, and cleavage steps were repeated for all rounds to complete the MERFISH imaging.

3D MERFISH imaging analysis. MERFISH image analysis was performed using a customized version of MERlin, a Python-based MERFISH analysis pipeline.

First, tiling artifacts were minimized at the ~10% overlap of neighboring FOVs by stitching the nuclear channel using BigStitcher. The transformation was then applied to each FOV to adjust the relative position for each FOV. This allowed a seamless transition between the neighboring FOVs in the final dataset. Second, a content aware deep learning-based image restoration algorithm CSBdeep was used to enhance the quality of MERFISH images captured with short exposure times. Specifically, an individual model was trained for each MERFISH bit color channel (560 and 650) separately. To accomplish this, 50 image pairs were randomly selected for each color channel, each with low and high signal-over-noise (SNR) images. From these imaging pairs, overlapping 128×128 image patches were generated, and these patches were further divided into a training set (80%) and evaluation set (20%). Using the training dataset, a neural network was trained using the CSBdeep applying the following parameters: kernel size=3, training batch size=10, and training steps per epoch=50. As a training loss function, the average of absolute differences was used. The performance of the model was then evaluated using the reserved 20% dataset to determine the optimal model parameters. Third, the images taken during each imaging round were aligned based on the fiducial bead images, accounting for X-Y drift in the stage position relative to the first round of imaging. Fourth, a high-pass filter was then applied to the enhanced images for each FOV to remove cellular background. The filtered images were then deconvolved using 20 rounds of Lucy-Richardson deconvolution to tighten RNA spots, and low-pass filtered them to account for small movements in the apparent centroid of RNAs between imaging rounds. Individual RNA molecules were identified by pixel-based decoding algorithm. Briefly, barcodes were individually assigned to each pixel, then aggregated adjacent pixels with the same barcodes into putative RNA molecules and filtered the list of putative RNA molecules to enrich for correctly identified transcripts. In detail, to assign each pixel to one of barcodes, the intensity vectors measured for each pixel were compared to the vectors corresponding to the valid barcodes. To aid comparison, the intensity of each image was normalized in a bit by median intensity across all FOVs in the bit to eliminate the intensity variation between hybridizations and color channels. After intensity normalization, intensity variations across pixels were further normalized by dividing the intensity vector for each pixel by its L2 norm. Similarly, each of the predesigned barcodes was normalized by its L2 norm. To assign a barcode to each pixel, the normalized barcode vector that was closest to the pixel's normalized intensity vector was identified. Pixels with the distance larger than 0.65 away from any valid barcode in the first step were excluded and any pixels with an intensity less than 10 were disregarded, as they are potentially off target binding probes or noise-induced artifacts amplified by the deep learning algorithm.

During the model training, two sets of confocal images of the same locations were used as input: one set taken with a short exposure time (100 ms) and the other set with a long exposure time (1 s). The short-exposure images was expected to contain high levels of noise. The long-exposure images were of higher quality hence lower noise level. These two images are taken at the same location should contain identical information despite of the different level of noise. The deep learning model was then trained to learn how to transform the low-quality, noisy low-exposure images into high-quality long-exposure images. The purpose of the deep learning model is to improve the quality of the image. The deep learning model was a convolutional neural network.

After assigning barcodes to each pixel independently, adjacent pixels that were assigned with the same barcodes were aggregated into putative RNA molecules, and then the list of putative RNA molecules was filtered to enrich for correctly identified transcripts for an overall barcode misidentification rate at 5%. Putative RNAs that contained only a single pixel were also removed, as they are prone to be background of spurious barcodes generated by random fluorescent fluctuations and had a substantially higher misidentification rate than those that contained 2 or more pixels. Finally, since the 3D MERFISH imaging samples at 1-micrometer intervals, it is possible to capture the same molecule in multiple z-sections. To avoid counting duplicate molecules, identical molecules present in adjacent z-sections were removed prior to assigning barcodes to individual cells.

3D cell segmentation. Cell segmentation was performed on the co-staining of DAPI and total mRNA using the deep learning-based cell segmentation algorithm, Cellpose 2.0. The segmentation model was fine-tuned with the user-in-the-loop approach of the Cellpose 2.0 using randomly selected z-slices containing the DAPI and polyA mRNA channels with the “CP” model as a starting point. After human-guided training, the segmentation model was applied to 3D z-stacks for each FOV to generate segmentation masks in 3D using cellpose 2.0 using its 3D mode. The cell boundaries were extracted for each cell and the cell boundaries exported as polygons.

Owing to an approximate 10% overlap in our field of view (FOV), it is possible that a single cell may be captured in two adjacent FOVs, resulting in duplicated cells. To address this issue, a method was developed to remove overlapping cells. Firstly, for each cell, its 10 nearest neighboring cells were identified, and for each of these neighbors, their overlapping volume was calculated. Next, the overlapping ratio between the two cells was determined by dividing the overlapping volume by the minimum volume of the two neighboring cells. The overlapping ratio ranged from 0% for cells with no overlap with any other cells to 100% for cells completely overlapping with another cell. Two cells were considered to be duplicates if their overlapping ratio is larger than 40% and, in this case, the cell of smaller volume is removed. This method allowed duplicated cells that appeared in multiple adjacent FOVs to be identified and removed. By implementing this method, the final dataset contained only unique cells, and any inaccuracies in downstream analysis could be avoided. The detected RNA molecules were assigned into the cell if the molecule position is within the boundaries of the cell to obtain the cell-by-gene matrix.

Unsupervised clustering analysis of 3D MERFISH data. After obtaining the cell-by-gene matrix as described above, preprocessing on the matrix was performed using the following steps. Firstly, cells that were potentially artifacts due to segmentation errors were removed. Specifically, cells with a small volume (<300 micrometer²), or low RNA count number (<30), or those captured in 1 micrometer z-sections fewer than 5 or more than 40 times, were excluded. These criteria were selected to exclude cells with low quality or insufficient information. Next, ~10% cells were removed as putative doublets identified using doubletFinder.

After the above preprocessing steps, the single cell data was analyzed using Seurat as described below. The gene vector was normalized for each cell by dividing each cell by its total RNA counts sum and then multiply the resulting number with a constant number 10,000 to ensure all cells contain the same total RNA counts. Following this normalization, a log transformation was performed on the cell-by-gene matrix. The normalized single-cell expression profiles were z-scored, followed by dimensionality reduction by principal component analysis. The first 30 principal components were used and graph-based Louvain community detection performed in the 30 principal components space with nearest neighborhood size k=15 and resolution r=0.8 to identify clusters.

From the first round of clustering, excitatory neuronal, inhibitory neuronal and non-neuronal cell types were identified based on the expression of the canonical marker genes. To further refine the cell classification, the procedure of dimensionality reduction and clustering was repeated, as described above, for the excitatory and inhibitory neurons separately. For the identification of neuronal clusters, the following parameters k=15 and r=5 for inhibitory neurons and k=15 and r=3 for excitatory neurons were used.

TABLE 1
Imaging platforms used for specific data acquisition

| FIG. | Setup | Confocal | Objective |
|---|---|---|---|
| FIG. 4 | Setup 2 | Andor, CSU W1 | Olympus |
| | | | UPLSAPO60XS2 |
| | | | 60X NA = 1.3 |
| FIGS. 5A-5D | Setup 2 | Andor Dragonfly, | Nikon MRD77410 |
| | | ACC-CR-DFLY- | 40X NA = 1.15 |
| | | 202-40 | |
| FIG. 6 | Setup 1 | Andor Dragonfly, | Nikon MRD77410 |
| | | ACC-CR-DFLY- | 40X NA = 1.15 |
| | | 202-40 | |
| FIGS. 7A-7C | Setup 1 | Andor Dragonfly, | Olympus |
| | | ACC-CR-DFLY- | UPLSAPO60XW |
| | | 202-40 | 60X NA = 1.2 |
| FIG. 7D | Setup 2 | Andor, CSU W1 | Nikon MRD77410 |
| | | | 40X NA = 1.15 |
| FIG. 8C | Setup 2 | Andor, CSU W1 | Olympus |
| | | | UPLSAPO60XW |
| | | | 60X NA = 1.2 |
| FIG. 9 | Setup 1 | Andor Dragonfly, | Nikon MRD77410 |
| | | ACC-CR-DFLY- | 40X NA = 1.15 |
| | | 202-40 | |
| FIG. 10 | Setup 1 | Andor Dragonfly, | Nikon MRD77410 |
| | | ACC-CR-DFLY- | 40X NA = 1.15 |
| | | 202-40 | |
| FIG. 11 | Setup 1 | Andor Dragonfly, | Nikon MRD77410 |
| | | ACC-CR-DFLY- | 40X NA = 1.15 |
| | | 202-40 | |
| FIG. 12 | Setup 1 | Andor Dragonfly, | Nikon MRD77410 |
| | | ACC-CR-DFLY- | 40X NA = 1.15 |
| | | 202-40 | |
| FIG. 1 | Setup 2 | Andor, CSU W1 | Olympus |
| | | | UPLSAPO60XS2 |
| | | | 60X NA = 1.3 |
| FIG. 2 | Setup 1 | Andor Dragonfly, | Nikon MRD77410 |
| | | ACC-CR-DFLY- | 40X NA = 1.15 |
| | | 202-40 | |
| FIG. 3 | Setup 1 | Andor Dragonfly, | Nikon MRD77410 |
| | | ACC-CR-DFLY- | 40X NA = 1.15 |
| | | 202-40 | |

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
using a confocal microscope to acquire images of a sample; and
determining nucleic acids within the sample using MERFISH.

2. The method of claim 1, further comprising acquiring images in more than one focal plane.

3. The method of claim 2, comprising acquiring images of focal planes that are at least 1 micrometer apart in a z-axis.

4. The method of any previous claim, wherein the confocal microscope is a spinning disk confocal microscope.

5. The method of any previous claim, further comprising using machine learning to enhance the images of the sample.

6. The method of any previous claim, further comprising executing a machine learning model, trained on confocal images, to accept an input image and output an enhanced images based on at least signal-to-noise ratio.

7. The method of any previous claim, further comprising executing a machine learning model, trained on confocal images, to accept an input image and output an enhanced images based on at least signal-to-background ratio.

8. The method of any previous claim, further comprising executing a machine learning model, trained on confocal image properties, to accept an input image and output an enhanced images based on at least signal-to-noise ratio.

9. The method of any previous claim, further comprising executing a machine learning model, trained on confocal image properties, to accept an input image and output an enhanced images based on at least signal-to-background ratio.

10. The method of any previous claim, wherein the sample has a thickness of at least 100 micrometers.

11. A method, comprising:
using a confocal microscope to acquire images of a sample; and
determining nucleic acids within the sample in 3 dimensions using the images.

12. The method of claim 11, further comprising using machine learning to enhance the images of the sample.

13. The method of claim 11 or 12, wherein the confocal microscope is a spinning disk confocal microscope.

14. A method, comprising:
exposing a sample to a plurality of nucleic acid probes;
for each of the nucleic acid probes, determining binding of the nucleic acid probes within the sample by acquiring images of the sample using a confocal microscope;
creating codewords based on the binding of the nucleic acid probes; and
for at least some of the codewords, matching the codeword to a valid codeword wherein, if no match is found, applying error correction to the codeword to form a valid codeword.

15. The method of claim 14, wherein the confocal microscope is a spinning disk confocal microscope.

16. A method, comprising:
using a confocal microscope to acquire images of a sample;
enhancing the images using machine learning; and
determining nucleic acids within the sample using MERFISH.

17. The method of claim 16, wherein the confocal microscope is a spinning disk confocal microscope.

18. A method, comprising:
using a confocal microscope to acquire images of a sample;
enhancing the images using machine learning; and
determining nucleic acids within the sample.

19. The method of claim 18, wherein the confocal microscope is a spinning disk confocal microscope.

20. A method, comprising:
using a confocal microscope to acquire images of a sample; and
determining nucleic acids within the sample by exposing the sample to a plurality of nucleic acid probes and determining binding of the plurality of the nucleic acid probes to the sample.

21. The method of claim 20, wherein the confocal microscope is a spinning disk confocal microscope.

22. A method, comprising:
using a confocal microscope to acquire images of a sample;
enhancing the images using machine learning; and
determining nucleic acids within the sample by exposing the sample to a plurality of nucleic acid probes and determining binding of the plurality of the nucleic acid probes to the sample.

23. The method of claim 22, wherein the confocal microscope is a spinning disk confocal microscope.

24. A method, comprising:
acquiring a plurality of images of the sample contained in a gel using a spinning disk confocal microscope, wherein between the acquisition of images, the gel exhibits a linear expansion of no more than 5%;
determining nucleic acids within the sample using MERFISH.

25. The method of claim 24, wherein the gel exhibits a linear expansion of no more than 2%.

26. A method, comprising:
acquiring a plurality of images of the sample contained in a gel using a spinning disk confocal microscope, wherein between the acquisition of images, the gel exhibits a linear expansion of no more than 5%;
determining nucleic acids within the sample; and
using machine learning to enhance the images of the sample.

27. The method of claim 26, wherein the gel exhibits a linear expansion of no more than 2%.
